(19) United States
(12) Patent Application Publication
(10) Pub. No.: US 2025/0230235 A1
(43) Pub. Date: Jul. 17, 2025

(54) TREATMENT OF MAST CELL RELATED DISORDERS

(71) Applicant: NOVELTY NOBILITY INC., Seongnam-si, Gyeonggi-do (KR)

(72) Inventors: Sang Gyu PARK, Gyeonggi-do (KR); Kwang-Hyeok KIM, Gyeonggi-do (KR)

(73) Assignee: NOVELTY NOBILITY INC., Seongnam-si, Gyeonggi-do (KR)

(21) Appl. No.: 18/698,982

(22) PCT Filed: Oct. 6, 2022
(86) PCT No.: PCT/KR2022/015064

§ 371 Date: Apr. 5, 2024

(30) Foreign Application Priority Data
Oct. 7, 2021 (KR) 10-2021-0133123

(51) Int. Cl. C07K 16/28; A61K 39/00; A61P 37/00

(52) U.S. Cl. CPC C07K 16/2803; A61P 37/00; A61K 2039/505; C07K 2317/34; C07K 2317/73; C07K 2317/76; C07K 2317/92

(57) ABSTRACT

Provided herein are methods and uses involving an anti-c-Kit antibody or an antigen binding fragment thereof for treating, preventing, managing or ameliorating mast cell related disorders.

### TECHNICAL FIELD

The present disclosure relates to treatment of mast cell related disorders.

### BACKGROUND ART

Mast cells are immune cells found in connective tissues throughout the body. Mast cells play a protective role in infectious disease against bacterial, viral, and parasitic in-fections through multiple mechanisms. Despite the protective role of mast cells, excessive proliferation and abnormal activation cause various mast cell diseases. Most mast cell diseases are caused by IgE-mediated degranulation. Allergic diseases, including, e.g., asthma, chronic spontaneous urticaria (CSU), allergic rhinitis (AR), atopic dermatitis (AD), food allergies, and anaphylaxis, are the most common mast cell diseases.

Anti-IgE monoclonal antibodies (e.g., omalizumab and ligelizumab), that inhibit mast cell degranulation, have been developed and approved by the Food and Drug Administration for the treatment of allergic asthma and CSU. The mechanism of action of omalizumab is to inhibit degranulation by neutralizing circulating IgE and reducing FcεRI expression. However, omalizumab has its limitations, as it cannot reduce the number of mast cells, which can be the fundamental cause of mast cell diseases (See, e.g., Beck L A, et al., J Allergy Clin Immunol 114:527-30). Therefore, repeated and continuous administration at 2 or 4 weeks interval is necessary for omalizumab treatment (subcutaneously 150-300 mg every 4 weeks or 225-375 mg every 2 weeks, depending on IgE level and body weight) (See, e.g., Easthope S et al., (2001) Drugs 61:253-60). This is also the reason why omalizumab has insufficient efficacy in mastocytosis and mast cell activation syndrome (MCAS) (See, e.g., Weiler C R (2019) J Allergy Clin Immunol Pract 7:2396-2397).

As a result, there is need for therapies that effectively manage or treat mast cell related disorders.

### DISCLOSURE OF INVENTION

### Technical Problem

To overcome the limitations of anti-IgE antibodies, the present disclosure aimed to develop therapeutic antibody that can inhibit both the proliferation and degranulation of mast cells. The present disclosure shows that antibodies that bind to specific domains of human c-Kit inhibit proliferation and migration of mast cells and the modulation of cytokine secretion by stem cell factor (SCF).

In one embodiment, the present disclosure is related to a treatment, prevention, management, or amelioration of mast cell-related disorders and/or one or more symptoms thereof, using an anti-c-Kit antibody that specifically binds to immunoglobulin-like (“Ig-like”) domains D2 and D3 of human c-Kit.

### Solution to Problem

In one embodiment, the present disclosure is related to a method of treating, preventing, managing or ameliorating one or more mast cell related disorders in a subject, comprising administering to a subject in need thereof a therapeutically effective amount of an anti-c-Kit antibody which specifically binds to a human c-Kit epitope, or an antigen binding fragment thereof.

In another embodiment, the present disclosure is related to an anti-c-Kit antibody which specifically binds to a human c-Kit epitope, or an antigen binding fragment thereof, for use in a method of treating, preventing, managing or ameliorating one or more mast cell related disorders in a subject.

In another embodiment, the present disclosure is related to use of an anti-c-Kit antibody which specifically binds to a human c-Kit epitope, or an antigen binding fragment thereof, for manufacture of a medicament for treating, preventing, managing or ameliorating one or more mast cell related disorders in a subject.

In another embodiment, the present disclosure is related to a pharmaceutical composition for treating, preventing, managing or ameliorating one or more mast cell related disorders in a subject, comprising an anti-c-Kit antibody which specifically binds to a human c-Kit epitope, or an antigen binding fragment thereof.

In one embodiment, the human c-Kit epitope may comprise an amino acid sequence of SEQ ID NO: 13.

In one embodiment, the anti-c-Kit antibody or the antigen binding fragment thereof may specifically bind to a human c-Kit epitope comprising an amino acid sequence of SEQ ID Nos: 11 and/or 12.

In one embodiment, the anti-c-Kit antibody or the antigen binding fragment thereof may specifically bind to at least one of R122, Y125, R181, K203, R205, S261 and H263 on amino acid sequence of SEQ ID NO: 14.

In one embodiment, the anti-c-Kit antibody or the antigen binding fragment thereof may specifically bind to c-Kit with equilibrium dissociation constant (KD) value less than or equal to 10⁻⁸ M, 10⁻⁹ M, 10⁻¹⁰ M, 10⁻¹¹ M, 8×10⁻¹² M, 6×10⁻¹² M, 4×10⁻¹² M, 3×10⁻¹² M, 2×10⁻¹² M, 1×10⁻¹² M, or 10⁻¹³ M.

In one embodiment, the anti-c-Kit antibody or the antigen binding fragment thereof specifically binds to c-Kit with binding affinity which is more than 50-fold stronger, 100-fold stronger, 150-fold stronger, 200-fold stronger, 250-fold stronger, 300-fold stronger, 350-fold stronger, 400-fold stronger, 450-fold stronger, 500-fold stronger, 550-fold stronger, or 600-fold stronger than that between SCF and c-Kit.

In one embodiment, the anti-c-Kit antibody or the antigen binding fragment thereof may cross-compete for binding to a human c-Kit epitope on an amino acid sequence of SEQ ID Nos: 11 and 12 with a reference antibody comprising VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

In one embodiment, the anti-c-Kit antibody or the antigen binding fragment thereof may comprise VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6, or comprise a variant of the CDRs, wherein the variant of the CDRs may comprise modification, deletion or substitution of one, two, three, four, or five amino acid(s) present in at least one CDR.

In one embodiment, In one embodiment, the anti-c-Kit antibody or the antigen binding fragment thereof may comprise VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

In one embodiment, the anti-c-Kit antibody or the antigen binding fragment thereof may comprise a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 7 and a VL comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 8.

In one embodiment, the anti-c-Kit antibody or the antigen binding fragment thereof may comprise a VH comprising SEQ ID NO: 7 and a VL comprising SEQ ID NO: 8.

In one embodiment, the anti-c-Kit antibody or the antigen binding fragment thereof may comprise a heavy chain comprising SEQ ID NO: 9 and a light chain comprising SEQ ID NO: 10.

In one embodiment, the anti-c-Kit antibody may be a bivalent monospecific antibody.

In one embodiment, the anti-c-Kit antibody may be a humanized antibody.

In one embodiment, the anti-c-Kit antibody may be a human antibody.

In one embodiment, the anti-c-Kit antibody may be a naked antibody.

In one embodiment, the subject may be a human.

In one embodiment, the therapeutically effective amount of the anti-c-Kit antibody or the antigen binding fragment thereof may be about 0.01 mg/kg to 1,000 mg/kg.

In one embodiment, the mast cell related disorders may be selected from the group consisting of mast cell activation syndrome (MCAS), primary mast cell activation syndrome, monoclonal mast cell activation syndrome (MMAS), idiopathic mast cell activation syndrome, secondary mast cell activation syndrome, asthma, allergic rhinitis, allergic inflammation, food allergies, rheumatoid arthritis, inflammatory bowel disease (IBD), multiple sclerosis, anaphylaxis, idiopathic anaphylaxis, Ig-E mediated anaphylaxis, non-Ig-E mediated anaphylaxis, neurofibromatosis, atopic dermatitis, psoriasis, urticaria, idiopathic urticaria, chronic urticaria, chronic spontaneous urticaria, allergic urticaria, idiopathic urticaria, cold induced urticaria, heat induced urticaria, dermatographic urticaria, vibratory urticaria, cholinergic urticaria, contact urticaria, symptomatic dermatographism, normo complementemic urticarial vasculitis, hypereosinophilic syndrome, fibrosis, idiopathic pulmonary fibrosis (IPF), pulmonary fibrosis, hepatic fibrosis, cardiac fibrosis, scleroderma, myelofibrosis, inflammatory conditions, pulmonary arterial hypertension (PAH), irritable bowel syndrome (IBS), dermatosis, mast cell activation disorder (MCAD), mastocytosis, cutaneous mastocytosis (e.g., urticaria pigmentosa), indolent systemic mastocytosis (ISM), smoldering systemic mastocytosis (SSM), mastocytosis with an associated hematologic neoplasm (SM-AHN), aggressive systemic mastocytosis (ASM), mast cell leukemia (MCL), mast cell sarcoma, systemic mastocytosis (SM), advanced SM (AdvSM), non-advanced SM (non-Adv SM), swelling (angioedema), primary pulmonary hypertension (PPH), mastocytic gastroenteritis, mastocytic colitis, pruritus, chronic pruritis, pruritis secondary to chronic kidney failure and heart, heart, vascular, intestinal, brain, kidney, liver, pancreas, muscle, bone and skin conditions associated with mast cells, autoimmune diseases, respiratory diseases, allergic diseases (including food allergies), allergic sinusitis, allergic contact dermatitis, erythema nodosum, erythema multiforme, cutaneous necrotizing venulitis and insect bite skin inflammation, bronchial asthma, inflammatory diseases, diabetes, type I diabetes, type II diabetes, central nervous system (CNS) disorders, interstitial cystitis and hematologic disorders.

In one embodiment, the mast cell related disorders may be selected from the group consisting of chronic spontaneous urticaria, cold induced urticaria, symptomatic dermatographism and normo complementemic urticarial vasculitis.

In one embodiment, a route of administration of the anti-c-Kit antibody or the antigen binding fragment thereof may be at least one of oral administration, subcutaneous administration, intramuscular administration intravenous administration, epidural administration, enteric administration, intracerebral administration, nasal administration, intraarterial administration, intracardiac administration, intraosseous infusion, intrathecal administration, and intraperitoneal administration.

Presented, below, are non-limiting embodiments of the pharmaceuticals, methods, pharmaceutical compositions, antibodies for use, and/or the use of antibodies described herein:

1. A method of treating, preventing, managing or ameliorating one or more mast cell related disorders in a subject, comprising administering to a subject in need thereof a therapeutically effective amount of an anti-c-Kit antibody which specifically binds to human c-Kit epitope comprising an amino acid sequence of SEQ ID NO: 13, or an antigen binding fragment thereof.

2. The method of embodiment 1, wherein the anti-c-Kit antibody or the antigen binding fragment thereof specifically binds to human c-Kit epitope comprising an amino acid sequence of SEQ ID Nos: 11 or 12.

3. The method of embodiment 1 or 2, wherein the anti-c-Kit antibody or the antigen binding fragment thereof specifically binds to at least one of R122, Y125, R181, K203, R205, S261 and H263 on amino acid sequence of SEQ ID NO: 14.

4. The method of any one of embodiments 1 to 3, wherein the anti-c-Kit antibody or the antigen binding fragment thereof specifically binds to c-Kit with equilibrium dissociation constant (KD) value less than or equal to 10⁻⁸ M, 10⁻⁹ M, 10⁻¹⁰ M, 10⁻¹¹ M, 8×10⁻¹² M, 6×10⁻¹² M, 4×10⁻¹² M, 3×10⁻¹² M, 2×10⁻¹² M, 1×10⁻¹² M, or 10⁻¹³ M.

5. The method of any one of embodiments 1 to 4, wherein the anti-c-Kit antibody or the antigen binding fragment thereof specifically binds to c-Kit with binding affinity which is more than 50-fold stronger, 100-fold stronger, 150-fold stronger, 200-fold stronger, 250-fold stronger, 300-fold stronger, 350-fold stronger, 400-fold stronger, 450-fold stronger, 500-fold stronger, 550-fold stronger, or 600-fold stronger than that between SCF and c-Kit.

6. The method of any one of embodiments 1 to 5, wherein the anti-c-Kit antibody or the antigen binding fragment thereof cross-competes for binding to human c-Kit epitope on an amino acid sequence of SEQ ID Nos: 11 and 12 with a reference antibody comprising VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

7. The method of any one of embodiments 1 to 6, wherein the anti-c-Kit antibody or the antigen binding fragment thereof comprises VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6, or comprises a variant of the CDRs, wherein the variant of the CDRs comprises modification, deletion or substitution of one, two, three, four, or five amino acid(s) present in at least one CDR.

8. The method of any one of embodiments 1 to 7, wherein the anti-c-Kit antibody or the antigen binding fragment thereof comprises VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

9. The method of any one of embodiments 1 to 8, wherein the anti-c-Kit antibody or the antigen binding fragment thereof comprises a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 7 and a VL comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 8.

10. The method of any one of embodiments 1 to 9, wherein the anti-c-Kit antibody or the antigen binding fragment thereof comprises a VH comprising SEQ ID NO: 7 and a VL comprising SEQ ID NO: 8.

11. The method of any one of embodiments 1 to 10, wherein the anti-c-Kit antibody or the antigen binding fragment thereof may comprise a heavy chain comprising SEQ ID NO: 9 and a light chain comprising SEQ ID NO: 10.

12. The method of any one of embodiments 1 to 11, wherein the anti-c-Kit antibody is a bivalent monospecific antibody.

13. The method of any one of embodiments 1 to 12, wherein the anti-c-Kit antibody is a humanized antibody.

14. The method of any one of embodiments 1 to 13, wherein the anti-c-Kit antibody is a naked antibody.

15. The method of any one of embodiments 1 to 14, wherein the subject is a human.

16. The method of any one of embodiments 1 to 15, wherein the therapeutically effective amount of the anti-c-Kit antibody or the antigen binding fragment thereof is about 0.01 mg/kg to 1,000 mg/kg.

17. The method of any one of embodiments 1 to 16, wherein the mast cell related disorders are selected from the group consisting of mast cell activation syndrome (MCAS), primary mast cell activation syndrome, monoclonal mast cell activation syndrome (MMAS), idiopathic mast cell activation syndrome, secondary mast cell activation syndrome, asthma, allergic rhinitis, allergic inflammation, food allergies, rheumatoid arthritis, inflammatory bowel disease (IBD), multiple sclerosis, anaphylaxis, idiopathic anaphylaxis, Ig-E mediated anaphylaxis, non-Ig-E mediated anaphylaxis, neurofibromatosis, atopic dermatitis, psoriasis, urticaria, idiopathic urticaria, chronic urticaria, chronic spontaneous urticaria, allergic urticaria, idiopathic urticaria, cold induced urticaria, heat induced urticaria, dermatographic urticaria, vibratory urticaria, cholinergic urticaria, contact urticaria, symptomatic dermatographism, normo complementemic urticarial vasculitis, hypereosinophilic syndrome, fibrosis, idiopathic pulmonary fibrosis (IPF), pulmonary fibrosis, hepatic fibrosis, cardiac fibrosis, scleroderma, myelofibrosis, inflammatory conditions, pulmonary arterial hypertension (PAH), irritable bowel syndrome (IBS), dermatosis, mast cell activation disorder (MCAD), mastocytosis, cutaneous mastocytosis (e.g., urticaria pigmentosa), indolent systemic mastocytosis (ISM), smoldering systemic mastocytosis (SSM), mastocytosis with an associated hematologic neoplasm (SM-AHN), aggressive systemic mastocytosis (ASM), mast cell leukemia (MCL), mast cell sarcoma, systemic mastocytosis (SM), advanced SM (AdvSM), non-advanced SM (non-Adv SM), swelling (angioedema), primary pulmonary hypertension (PPH), mastocytic gastroenteritis, mastocytic colitis, pruritus, chronic pruritis, pruritis secondary to chronic kidney failure and heart, heart, vascular, intestinal, brain, kidney, liver, pancreas, muscle, bone and skin conditions associated with mast cells, autoimmune diseases, respiratory diseases, allergic diseases (including, for example. food allergies), allergic sinusitis, allergic contact dermatitis, erythema nodosum, erythema multiforme, cutaneous necrotizing venulitis and insect bite skin inflammation, bronchial asthma, inflammatory diseases, diabetes, type I diabetes, type II diabetes, central nervous system (CNS) disorders, interstitial cystitis and hematologic disorders.

18. The method of any one of embodiments 1 to 17, wherein the mast cell related disorders are selected from the group consisting of chronic spontaneous urticaria, cold induced urticaria, symptomatic dermatographism and normo complementemic urticarial vasculitis.

19. The method of any one of embodiments 1 to 18, a route of administration of the anti-c-Kit antibody or the antigen binding fragment thereof may be at least one of oral administration, subcutaneous administration, intramuscular administration intravenous administration, epidural administration, enteric administration, intracerebral administration, nasal administration, intraarterial administration, intracardiac administration, intraosseous infusion, intrathecal administration, and intraperitoneal administration.

20. A pharmaceutical composition for treating, preventing, managing or ameliorating one or more mast cell related disorders in a subject, comprising an anti-c-Kit antibody which specifically binds to human c-Kit epitope comprising an amino acid sequence of SEQ ID NO: 13, or an antigen binding fragment thereof.

21. The pharmaceutical composition of embodiment 20, wherein the antibody or the antigen binding fragment thereof specifically binds to human c-Kit epitope comprising an amino acid sequence of SEQ ID Nos: 11 or 12.

22. The pharmaceutical composition of embodiment 20 or 21, wherein the antibody or the antigen binding fragment thereof specifically binds to at least one of R122, Y125, R181, K203, R205, S261 and H263 on amino acid sequence of SEQ ID NO: 14.

23. The pharmaceutical composition of any one of embodiments 20 to 22, wherein the antibody or the antigen binding fragment thereof specifically binds to c-Kit with equilibrium dissociation constant (KD) value less than or equal to 10⁻⁸ M, 10⁻⁹ M, 10⁻¹⁰ M, 10⁻¹¹ M, 8×10⁻¹² M, 6×10⁻¹² M, 4×10⁻¹² M, 3×10⁻¹² M, 2×10⁻¹² M, 1×10⁻¹² M, or 10⁻¹³ M.

24. The pharmaceutical composition of any one of embodiments 20 to 23, wherein the antibody or the antigen binding fragment thereof specifically binds to c-Kit with binding affinity which is more than 50-fold stronger, 100-fold stronger, 150-fold stronger, 200-fold stronger, 250-fold stronger, 300-fold stronger, 350-fold stronger, 400-fold stronger, 450-fold stronger, 500-fold stronger, 550-fold stronger, or 600-fold stronger than that between SCF and c-Kit.

25. The pharmaceutical composition of any one of embodiments 20 to 24, wherein the antibody or the antigen binding fragment thereof cross-competes for binding to human c-Kit epitope on an amino acid sequence of SEQ ID Nos: 11 and 12 with a reference antibody comprising VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

26. The pharmaceutical composition of any one of embodiments 20 to 25, wherein the antibody or the antigen binding fragment thereof comprises VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6, or comprises a variant of the CDRs, wherein the variant of the CDRs comprises modification, deletion or substitution of one, two, three, four, or five amino acid(s) present in at least one CDR.

27. The pharmaceutical composition of any one of embodiments 20 to 26, wherein the antibody or the antigen binding fragment thereof comprises VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

28. The pharmaceutical composition of any one of embodiments 20 to 27, wherein the antibody or the antigen binding fragment thereof comprises a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 7 and a VL comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 8.

29. The pharmaceutical composition of any one of embodiments 20 to 28, wherein the antibody or the antigen binding fragment thereof comprises a VH comprising SEQ ID NO: 7 and a VL comprising SEQ ID NO: 8.

30. The pharmaceutical composition of any one of embodiments 20 to 29, wherein the anti-c-Kit antibody or the antigen binding fragment thereof may comprise a heavy chain comprising SEQ ID NO: 9 and a light chain comprising SEQ ID NO: 10.

31. The pharmaceutical composition of any one of embodiments 20 to 30, wherein the antibody is a bivalent monospecific antibody.

32. The pharmaceutical composition of any one of embodiments 20 to 31, wherein the antibody is a humanized antibody.

33. The pharmaceutical composition of any one of embodiments 20 to 32, wherein the antibody is a naked antibody.

34. The pharmaceutical composition of any one of embodiments 20 to 33, wherein the subject is a human.

35. The pharmaceutical composition of any one of embodiments 20 to 34, wherein an effective amount of the anti-c-Kit antibody or the antigen binding fragment thereof is about 0.01 mg/kg to 1,000 mg/kg.

36. The pharmaceutical composition of any one of embodiments 20 to 35, wherein the mast cell related disorders are selected from the group consisting of mast cell activation syndrome (MCAS), primary mast cell activation syndrome, monoclonal mast cell activation syndrome (MMAS), idiopathic mast cell activation syndrome, secondary mast cell activation syndrome, asthma, allergic rhinitis, allergic inflammation, food allergies, rheumatoid arthritis, inflammatory bowel disease (IBD), multiple sclerosis, anaphylaxis, idiopathic anaphylaxis, Ig-E mediated anaphylaxis, non-Ig-E mediated anaphylaxis, neurofibromatosis, atopic dermatitis, psoriasis, urticaria, idiopathic urticaria, chronic urticaria, chronic spontaneous urticaria, allergic urticaria, idiopathic urticaria, cold induced urticaria, heat induced urticaria, dermatographic urticaria, vibratory urticaria, cholinergic urticaria, contact urticaria, symptomatic dermatographism, normo complementemic urticarial vasculitis, hypereosinophilic syndrome, fibrosis, idiopathic pulmonary fibrosis (IPF), pulmonary fibrosis, hepatic fibrosis, cardiac fibrosis, scleroderma, myelofibrosis, inflammatory conditions, pulmonary arterial hypertension (PAH), irritable bowel syndrome (IBS), dermatosis, mast cell activation disorder (MCAD), mastocytosis, cutaneous mastocytosis (e.g., urticaria pigmentosa), indolent systemic mastocytosis (ISM), smoldering systemic mastocytosis (SSM), mastocytosis with an associated hematologic neoplasm (SM-AHN), aggressive systemic mastocytosis (ASM), mast cell leukemia (MCL), mast cell sarcoma, systemic mastocytosis (SM), advanced SM (AdvSM), non-advanced SM (non-Adv SM), swelling (angioedema), primary pulmonary hypertension (PPH), mastocytic gastroenteritis, mastocytic colitis, pruritus, chronic pruritis, pruritis secondary to chronic kidney failure and heart, heart, vascular, intestinal, brain, kidney, liver, pancreas, muscle, bone and skin conditions associated with mast cells, autoimmune diseases, respiratory diseases, allergic diseases (including, for example. food allergies), allergic sinusitis, allergic contact dermatitis, erythema nodosum, erythema multiforme, cutaneous necrotizing venulitis and insect bite skin inflammation, bronchial asthma, inflammatory diseases, diabetes, type I diabetes, type II diabetes, central nervous system (CNS) disorders, interstitial cystitis and hematologic disorders.

37. The pharmaceutical composition of any one of embodiments 20 to 36, wherein the mast cell related disorders are selected from the group consisting of chronic spontaneous urticaria, cold induced urticaria, symptomatic dermatographism and normo complementemic urticarial vasculitis.

38. The pharmaceutical composition of any one of embodiments 20 to 37, a route of administration of the anti-c-Kit antibody or the antigen binding fragment thereof may be at least one of oral administration, subcutaneous administration, intramuscular administration intravenous administration, epidural administration, enteric administration, intracerebral administration, nasal administration, intraarterial administration, intracardiac administration, intraosseous infusion, intrathecal administration, and intraperitoneal administration.

39. An anti-c-Kit antibody which specifically binds to human c-Kit epitope comprising an amino acid sequence of SEQ ID NO: 13, or an antigen binding fragment thereof, for use in a method of treating, preventing, managing or ameliorating one or more mast cell related disorders in a subject.

40. The anti-c-Kit antibody or the antigen binding fragment thereof for use of embodiment 39, wherein the anti-c-Kit antibody or the antigen binding fragment thereof specifically binds to human c-Kit epitope comprising an amino acid sequence of SEQ ID Nos: 11 or 12.

41. The anti-c-Kit antibody or the antigen binding fragment thereof for use of embodiment 39 or 40, wherein the anti-c-Kit antibody or the antigen binding fragment thereof specifically binds to at least one of R122, Y125, R181, K203, R205, S261 and H263 on amino acid sequence of SEQ ID NO: 14.

42. The anti-c-Kit antibody or the antigen binding fragment thereof for use of any one of embodiments 39 to 41, wherein the anti-c-Kit antibody or the antigen binding fragment thereof specifically binds to c-Kit with equilibrium dissociation constant (KD) value less than or equal to 10⁻⁸ M, 109 M, 10⁻¹⁰ M, 10⁻¹¹ M, 8×10⁻¹² M, 6×10⁻¹² M, 4×10⁻¹² M, 3×10⁻¹² M, 2×10⁻¹² M, 1×10⁻¹² M, or 10⁻¹³ M.

43. The anti-c-Kit antibody or the antigen binding fragment thereof for use of any one of embodiments 39 to 42, wherein the anti-c-Kit antibody or the antigen binding fragment thereof specifically binds to c-Kit with binding affinity which is more than 50-fold stronger, 100-fold stronger, 150-fold stronger, 200-fold stronger, 250-fold stronger, 300-fold stronger, 350-fold stronger, 400-fold stronger, 450-fold stronger, 500-fold stronger, 550-fold stronger, or 600-fold stronger than that between SCF and c-Kit.

44. The anti-c-Kit antibody or the antigen binding fragment thereof for use of any one of embodiments 39 to 43, wherein the anti-c-Kit antibody or the antigen binding fragment thereof cross-competes for binding to human c-Kit epitope on an amino acid sequence of SEQ ID Nos: 11 and 12 with a reference antibody comprising VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

45. The anti-c-Kit antibody or the antigen binding fragment thereof for use of any one of embodiments 39 to 44, wherein the anti-c-Kit antibody or the antigen binding fragment thereof comprises VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6, or comprises a variant of the CDRs, wherein the variant of the CDRs comprises modification, deletion or substitution of one, two, three, four, or five amino acid(s) present in at least one CDR.

46. The anti-c-Kit antibody or the antigen binding fragment thereof for use of any one of embodiments 39 to 45, wherein the anti-c-Kit antibody or the antigen binding fragment thereof comprises VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

47. The anti-c-Kit antibody or the antigen binding fragment thereof for use of any one of embodiments 39 to 46, wherein the anti-c-Kit antibody or the antigen binding fragment thereof comprises a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 7 and a VL comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 8.

48. The anti-c-Kit antibody or the antigen binding fragment thereof for use of any one of embodiments 39 to 47, wherein the anti-c-Kit antibody or the antigen binding fragment thereof comprises a VH comprising SEQ ID NO: 7 and a VL comprising SEQ ID NO: 8.

49. The anti-c-Kit antibody or the antigen binding fragment thereof for use of any one of embodiments 39 to 48, wherein the anti-c-Kit antibody or the antigen binding fragment thereof may comprise a heavy chain comprising SEQ ID NO: 9 and a light chain comprising SEQ ID NO: 10.

50. The anti-c-Kit antibody or the antigen binding fragment thereof for use of any one of embodiments 39 to 49, wherein the anti-c-Kit antibody is a bivalent monospecific antibody.

51. The anti-c-Kit antibody or the antigen binding fragment thereof for use of any one of embodiments 39 to 50, wherein the anti-c-Kit antibody is a humanized antibody.

52. The anti-c-Kit antibody or the antigen binding fragment thereof for use of any one of embodiments 39 to 51, wherein the anti-c-Kit antibody is a naked antibody.

53. The anti-c-Kit antibody or the antigen binding fragment thereof for use of any one of embodiments 39 to 52, wherein the subject is a human.

54. The anti-c-Kit antibody or the antigen binding fragment thereof for use of any one of embodiments 39 to 53, wherein an effective amount of the anti-c-Kit antibody or the antigen binding fragment thereof is about 0.01 mg/kg to 1,000 mg/kg.

55. The anti-c-Kit antibody or the antigen binding fragment thereof for use of any one of embodiments 39 to 54, wherein the mast cell related disorders are selected from the group consisting of mast cell activation syndrome (MCAS), primary mast cell activation syndrome, monoclonal mast cell activation syndrome (MMAS), idiopathic mast cell activation syndrome, secondary mast cell activation syndrome, asthma, allergic rhinitis, allergic inflammation, food allergies, rheumatoid arthritis, inflammatory bowel disease (IBD), multiple sclerosis, anaphylaxis, idiopathic anaphylaxis, Ig-E mediated anaphylaxis, non-Ig-E mediated anaphylaxis, neurofibromatosis, atopic dermatitis, psoriasis, urticaria, idiopathic urticaria, chronic urticaria, chronic spontaneous urticaria, allergic urticaria, idiopathic urticaria, cold induced urticaria, heat induced urticaria, dermatographic urticaria, vibratory urticaria, cholinergic urticaria, contact urticaria, symptomatic dermatographism, normo complementemic urticarial vasculitis, hypereosinophilic syndrome, fibrosis, idiopathic pulmonary fibrosis (IPF), pulmonary fibrosis, hepatic fibrosis, cardiac fibrosis, scleroderma, myelofibrosis, inflammatory conditions, pulmonary arterial hypertension (PAH), irritable bowel syndrome (IBS), dermatosis, mast cell activation disorder (MCAD), mastocytosis, cutaneous mastocytosis (e.g., urticaria pigmentosa), indolent systemic mastocytosis (ISM), smoldering systemic mastocytosis (SSM), mastocytosis with an associated hematologic neoplasm (SM-AHN), aggressive systemic mastocytosis (ASM), mast cell leukemia (MCL), mast cell sarcoma, systemic mastocytosis (SM), advanced SM (AdvSM), non-advanced SM (non-Adv SM), swelling (angioedema), primary pulmonary hypertension (PPH), mastocytic gastroenteritis, mastocytic colitis, pruritus, chronic pruritis, pruritis secondary to chronic kidney failure and heart, heart, vascular, intestinal, brain, kidney, liver, pancreas, muscle, bone and skin conditions associated with mast cells, autoimmune diseases, respiratory diseases, allergic diseases (including, for example. food allergies), allergic sinusitis, allergic contact dermatitis, erythema nodosum, erythema multiforme, cutaneous necrotizing venulitis and insect bite skin inflammation, bronchial asthma, inflammatory diseases, diabetes, type I diabetes, type II diabetes, central nervous system (CNS) disorders, interstitial cystitis and hematologic disorders.

56. The anti-c-Kit antibody or the antigen binding fragment thereof for use of any one of embodiments 39 to 55, wherein the mast cell related disorders are selected from the group consisting of chronic spontaneous urticaria, cold induced urticaria, symptomatic dermatographism and normo complementemic urticarial vasculitis.

57. The anti-c-Kit antibody or the antigen binding fragment thereof for use of any one of embodiments 39 to 56, a route of administration of the anti-c-Kit antibody or the antigen binding fragment thereof may be at least one of oral administration, subcutaneous administration, intramuscular administration intravenous administration, epidural administration, enteric administration, intracerebral administration, nasal administration, intraarterial administration, intracardiac administration, intraosseous infusion, intrathecal administration, and intraperitoneal administration.

58. Use of an anti-c-Kit antibody which specifically binds to human c-Kit epitope comprising an amino acid sequence of SEQ ID NO: 13, or an antigen binding fragment thereof, for manufacture of a medicament for treating, preventing, managing or ameliorating one or more mast cell related disorders in a subject.

59. The use of the anti-c-Kit antibody or the antigen binding fragment thereof of embodiment 58, wherein the antibody or the antigen binding fragment thereof specifically binds to human c-Kit epitope comprising an amino acid sequence of SEQ ID Nos: 11 or 12.

60. The use of the anti-c-Kit antibody or the antigen binding fragment thereof of embodiment 58 or 59, wherein the antibody or the antigen binding fragment thereof specifically binds to at least one of R122, Y125, R181, K203, R205, S261 and H263 on amino acid sequence of SEQ ID NO: 14.

61. The use of the anti-c-Kit antibody or the antigen binding fragment thereof of any one of embodiments 58 to 60, wherein the antibody or the antigen binding fragment thereof specifically binds to c-Kit with equilibrium dissociation constant (KD) value less than or equal to 10⁻⁸ M, 10⁻⁹ M, 10⁻¹⁰ M, 10⁻¹¹ M, 8×10⁻¹² M, 6×10⁻¹² M, 4×10⁻¹² M, 3×10⁻¹² M, 2×10⁻¹² M, 1×10⁻¹² M, or 10⁻¹³ M.

62. The use of the anti-c-Kit antibody or the antigen binding fragment thereof of any one of embodiments 58 to 61, wherein the antibody or the antigen binding fragment thereof specifically binds to c-Kit with binding affinity which is more than 50-fold stronger, 100-fold stronger, 150-fold stronger, 200-fold stronger, 250-fold stronger, 300-fold stronger, 350-fold stronger, 400-fold stronger, 450-fold stronger, 500-fold stronger, 550-fold stronger, or 600-fold stronger than that between SCF and c-Kit.

63. The use of the anti-c-Kit antibody or the antigen binding fragment thereof of any one of embodiments 58 to 62, wherein the antibody or the antigen binding fragment thereof cross-competes for binding to human c-Kit epitope on an amino acid sequence of SEQ ID Nos: 11 and 12 with a reference antibody comprising VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

64. The use of the anti-c-Kit antibody or the antigen binding fragment thereof of any one of embodiments 58 to 63, wherein the antibody or the antigen binding fragment thereof comprises VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6, or comprises a variant of the CDRs, wherein the variant of the CDRs comprises modification, deletion or substitution of one, two, three, four, or five amino acid(s) present in at least one CDR.

65. The use of the anti-c-Kit antibody or the antigen binding fragment thereof of any one of embodiments 58 to 64, wherein the antibody or the antigen binding fragment thereof comprises VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and VL CDR3 of SEQ ID NO: 6.

66. The use of the anti-c-Kit antibody or the antigen binding fragment thereof of any one of embodiments 58 to 65, wherein the antibody or the antigen binding fragment thereof comprises a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 7 and a VL comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 8.

67. The use of the anti-c-Kit antibody or the antigen binding fragment thereof of any one of embodiments 58 to 66, wherein the antibody or the antigen binding fragment thereof comprises a VH comprising SEQ ID NO: 7 and a VL comprising SEQ ID NO: 8.

68. The use of the anti-c-Kit antibody or the antigen binding fragment thereof of any one of embodiments 58 to 67, wherein the anti-c-Kit antibody or the antigen binding fragment thereof may comprise a heavy chain comprising SEQ ID NO: 9 and a light chain comprising SEQ ID NO: 10.

69. The use of the anti-c-Kit antibody or the antigen binding fragment thereof of any one of embodiments 58 to 68, wherein the antibody is a bivalent monospecific antibody.

70. The use of the anti-c-Kit antibody or the antigen binding fragment thereof of any one of embodiments 58 to 69, wherein the antibody is a humanized antibody.

71. The use of the anti-c-Kit antibody or the antigen binding fragment thereof of any one of embodiments 58 to 70, wherein the antibody is a naked antibody.

72. The use of the anti-c-Kit antibody or the antigen binding fragment thereof of any one of embodiments 58 to 71, wherein the subject is a human.

73. The use of the anti-c-Kit antibody or the antigen binding fragment thereof of any one of embodiments 58 to 72, wherein an effective amount of the anti-c-Kit antibody or the antigen binding fragment thereof is about 0.01 mg/kg to 1,000 mg/kg.

74. The use of the anti-c-Kit antibody or the antigen binding fragment thereof of any one of embodiments 58 to 73, wherein the mast cell related disorders are selected from the group consisting of mast cell activation syndrome (MCAS), primary mast cell activation syndrome, monoclonal mast cell activation syndrome (MMAS), idiopathic mast cell activation syndrome, secondary mast cell activation syndrome, asthma, allergic rhinitis, allergic inflammation, food allergies, rheumatoid arthritis, inflammatory bowel disease (IBD), multiple sclerosis, anaphylaxis, idiopathic anaphylaxis, Ig-E mediated anaphylaxis, non-Ig-E mediated anaphylaxis, neurofibromatosis, atopic dermatitis, psoriasis, urticaria, idiopathic urticaria, chronic urticaria, chronic spontaneous urticaria, allergic urticaria, idiopathic urticaria, cold induced urticaria, heat induced urticaria, dermatographic urticaria, vibratory urticaria, cholinergic urticaria, contact urticaria, symptomatic dermatographism, normo complementemic urticarial vasculitis, hypereosinophilic syndrome, fibrosis, idiopathic pulmonary fibrosis (IPF), pulmonary fibrosis, hepatic fibrosis, cardiac fibrosis, scleroderma, myelofibrosis, inflammatory conditions, pulmonary arterial hypertension (PAH), irritable bowel syndrome (IBS), dermatosis, mast cell activation disorder (MCAD), mastocytosis, cutaneous mastocytosis (e.g., urticaria pigmentosa), indolent systemic mastocytosis (ISM), smoldering systemic mastocytosis (SSM), mastocytosis with an associated hematologic neoplasm (SM-AHN), aggressive systemic mastocytosis (ASM), mast cell leukemia (MCL), mast cell sarcoma, systemic mastocytosis (SM), advanced SM (AdvSM), non-advanced SM (non-Adv SM), swelling (angioedema), primary pulmonary hypertension (PPH), mastocytic gastroenteritis, mastocytic colitis, pruritus, chronic pruritis, pruritis secondary to chronic kidney failure and heart, heart, vascular, intestinal, brain, kidney, liver, pancreas, muscle, bone and skin conditions associated with mast cells, autoimmune diseases, respiratory diseases, allergic diseases (including, for example. food allergies), allergic sinusitis, allergic contact dermatitis, erythema nodosum, erythema multiforme, cutaneous necrotizing venulitis and insect bite skin inflammation, bronchial asthma, inflammatory diseases, diabetes, type I diabetes, type II diabetes, central nervous system (CNS) disorders, interstitial cystitis and hematologic disorders.

75. The use of the anti-c-Kit antibody or the antigen binding fragment thereof of any one of embodiments 58 to 74, wherein the mast cell related disorders are selected from the group consisting of chronic spontaneous urticaria, cold induced urticaria, symptomatic dermatographism and normo complementemic urticarial vasculitis.

76. The use of the anti-c-Kit antibody or the antigen binding fragment thereof of any one of embodiments 58 to 75, a route of administration of the anti-c-Kit antibody or the antigen binding fragment thereof may be at least one of oral administration, subcutaneous administration, intramuscular administration intravenous administration, epidural administration, enteric administration, intracerebral administration, nasal administration, intraarterial administration, intracardiac administration, intraosseous infusion, intrathecal administration, and intraperitoneal administration.

77. A pharmaceutical composition comprising the antibody or the antigen binding fragment thereof of any one of embodiments 39 to 57 and a pharmaceutically acceptable carrier.

78. A kit comprising the antibody or the antigen binding fragment thereof of any one of embodiments 39 to 57, or the pharmaceutical composition of any one of embodiments 20-38.

### Advantageous Effects of Invention

In one embodiment, the anti-c-Kit antibody of the present disclosure can inhibit c-Kit signaling (e.g., phosphorylation pathway of c-Kit) by blocking the binding of stem cell factor (SCF), also known as c-Kit ligand.

In one embodiment, the anti-c-Kit antibody of the present disclosure can suppress proliferation, migration, and degranulation in mast cells by inhibiting c-Kit signaling.

In one embodiment, the anti-c-Kit antibody of the present disclosure can suppress the secretion of pro-inflammatory cytokines, including and not limited to, granulocyte-macrophage colony-stimulating factor, vascular endothelial growth factor, C—C motif chemokine ligand 2, brain-derived neurotrophic factor, and complement component C5/C5a, which can exacerbate allergic symptoms.

In one embodiment, the anti-c-Kit antibody of the present disclosure can be used as a therapeutic agent for mast cell diseases.

### BRIEF DESCRIPTION OF DRAWINGS

FIG. 1 shows the flow cytometry result of 2G4 and 4C9 antibodies' binding in LAD2 cells. FIG. 1 demonstrates that 2G4 and 4C9 antibodies bind to c-Kit on the cell surface of LAD2 cells.

FIGS. 2a and 2b show the Western blot result of 2G4 and 4C9 antibodies' inhibition of c-Kit in LAD2 cells. FIGS. 2a and 2b demonstrate that 2G4 and 4C9 antibodies inhibit SCF-mediated c-Kit activation in LAD2 cells.

FIGS. 3a and 3b show the results of cell proliferation assay. FIGS. 3a and 3b demonstrate that 2G4 antibody inhibits cell proliferation in LAD2 cells. In FIGS. 3a and 3b, the black dashed line (100%) indicates normalized cell counts in the well without SCF and antibodies at 7 days.

FIG. 4 show the results of migration assay. FIG. 4 demonstrates that 2G4 antibody inhibits cell migration in LAD2 cells. All results represent the mean±SD of three independent experiments. * vs. SCF⁻/Antibody and # vs. SCF⁺/Antibody. * P<0.05, and #P<0.05 (Student's two-tailed t-test).

FIGS. 5a, 5b, 5c and 5d shows the results of mast cell degranulation assay. FIGS. 5a, 5b and 5c demonstrates that 2G4 antibody does not cause antibody-mediated mast cell degranulation in non-sensitized, IgE-sensitized, and IFN-γ-sensitized LAD cells, but 4C9 antibody does cause degranulation. FIG. 5d demonstrates that 2G4 antibody inhibits IgE-mediated degranulation enhanced by SCF. All results represent the mean±SD of three independent experiments. *, **, and *** vs Untreated; #, ##, and ### vs. SCF⁻/Streptavidin⁻; §, §§, and § § § vs. SCF⁺/Streptavidin⁻; †, ††, and ††† vs. SCF⁻/Streptavidin⁺; and ‡, ‡, and ‡‡‡ vs. SCF⁺/Streptavidin⁺. * P<0.05, ** P<0.01, and *** P<0.001; #P<0.05, ##P<0.01, and ###P<0.001; § P<0.05, §§ P<0.01, and § § § P<0.001; † P<0.05, †† P<0.01, and ††† P<0.001; and ‡ P<0.05, ‡‡ P<0.01, and ‡‡‡ P<0.001 (one-way ANOVA with Dunnett's post-test).

FIG. 6 shows the results of cytokine-release assay. FIG. 6 demonstrates that 2G4 antibody inhibits the modulation of cytokine secretion. In FIG. 6, the result represents the mean±SD of three independent experiments. *, **, and *** vs. IgE+Vehicle; #, ##, and ###vs. IgE+Streptavidin; §, § §, and § § § vs. IgE+Streptavidin/SCF. * P<0.05, ** P<0.01, and *** P<0.001; #P<0.05, ##P<0.01, and ###P<0.001; and § P<0.05, § § P<0.01, and § § § P<0.001 (Student's two-tailed t-test).

FIG. 7 shows the results of cytokine-release assay. FIG. 7 demonstrates that 2G4 inhibits modulation of cytokines secretion. Certain cytokines, including CCL5, M-CSF, and IL-2, were decreased by SCF and the decrease in cytokine release was inhibited by 2G4 (FIG. 7). *, **, and *** vs. IgE+Vehicle; #, ##, and ### vs. IgE+Streptavidin; §, § §, and § § § vs. IgE+Streptavidin/SCF. * P<0.05, ** P<0.01, and *** P<0.001; #P<0.05, ##P<0.01, and ###P<0.001; and § P<0.05, § § P<0.01, and § § § P<0.001 (Student's two-tailed t-test).

### MODE FOR THE INVENTION

Various embodiments or examples of the present disclosure are for the purpose of illustrating and/or explaining the present application and are not intended to be limiting. The present disclosure includes various modifications, equivalents and/or alternatives of each embodiment or example described in this application and/or any possible combinations of all or part of each embodiment or example described in this application. The scope of rights of the present disclosure is not limited to various embodiments or examples set forth below or the specific descriptions of the embodiments or examples.

All technical and scientific terms used in this application, unless otherwise defined, generally have the ordinary meanings understood by a person of ordinary skill in the art to which this disclosure pertains. All terms used in this application are chosen for the purpose of describing and/or explaining this disclosure and are not intended to limit the scope of rights under this disclosure. Certain terms are discussed in this application to provide additional guidance in describing and/or explaining the compositions and method of the present disclosure.

### I. Definitions

Whenever used in the present disclosure, the singular forms “a,” “an” and “the” and singular words include plural reference unless the context clearly dictates otherwise, and the same shall apply to the singular forms and words set forth in the claims. For example, “a compound” includes a plurality of such compounds, and “a compound A” includes a plurality of compounds A.

The term “and/or” means any one or more of the items, any combination of the items, or all of the items with which this term is associated. The terms “containing,” “contain,” “contains,” “including,” “include,” “includes,” “having,” “have,” “has,” “with” or variants thereof are interpreted as inclusive in a manner similar to the term “comprising.”

All ranges recited in this application may include any and all possible subranges and combinations of subranges thereof. A recited range includes each specific value, integer or decimal within the ranges. One of ordinary skill in the art may readily understand that any recited range sufficiently describes and/or enables its subranges, including and not limited to equal halves, thirds, quarters, fifths or tenths of the recited range.

The terms “greater than,” “more than,” “at least,” “or more,” “less than,” “up to” and the like, include the numbers recited, and the terms refer to ranges that may be broken down into subranges as discussed above. Specific values recited for ranges are for illustration purposes only and are not intended to be limiting, and they do not exclude other values within the ranges.

Unless indicated otherwise, the terms “about” and “approximately” generally include values proximate to the recited range or value within an acceptable degree of error, as well known to those skilled in the art. The acceptable degree of error may be determined in view of the nature or precision of the measurements. In one embodiment, the acceptable degree of error may be determined based on equivalence in terms of the functionality of the composition or method or the embodiment. In one embodiment, in the context of numerical values or ranges set forth in this disclosure, the term “about” or “approximately” can refer to a variation of ±25%, ±20%, ±15%, ±10%, ±9%, ±8%, ±7%, ±6%, ±5%, ±4%, ±3%, ±2% or ±1% of the value provided. In another embodiment, particularly in biological systems, the terms “about” and “approximately” may mean values that are within an order of magnitude, within 9-fold, 8-fold, 7-fold, 6-fold, 5-fold, 4-fold, 3-fold or 2-fold of a given value. All numerical quantities provided in this application are approximations unless stated otherwise. In addition, these quantities inherently contain variability necessarily resulting from their measurements.

As used herein, the terms “antibody” and “immunoglobulin” and “Ig” are terms of art and can be used interchangeably herein and refer to a molecule with an antigen binding site that immunospecifically binds an antigen.

As used herein, an “antigen” is a moiety or molecule that contains an epitope, and, as such, also is specifically bound by an antibody. In a specific embodiment, the antigen, to which an antibody described herein binds, is c-Kit (e.g., human c-Kit), or a fragment thereof, for example, an extracellular domain of c-Kit (e.g., human c-Kit) or a D2 and/or D3 region of c-Kit (e.g., human c-Kit).

As used herein, the terms “antigen binding domain,” “antigen binding region,” “antigen binding fragment,” and similar terms refer to a portion of an antibody molecule which comprises the amino acid residues that interact with an antigen and confer on the antibody molecule its specificity for the antigen (e.g., the comple-mentarity determining regions (CDRs)). The antigen binding region can be derived from any animal species, such as rodents (e.g., mouse, rat or hamster) and humans. The CDRs of an antibody molecule can be determined by any method well known to one of ordinary skill in the art.

As used herein, the term “constant region” or “constant domain” refers to an antibody portion, e.g., a carboxyl terminal portion of a light and/or heavy chain which is not directly involved in binding of an antibody to an antigen but which exhibits or contributes to various effector functions, such as interaction with the Fc receptor. The term refers to a portion of an immunoglobulin molecule having a generally more conserved amino acid sequence relative to an immunoglobulin variable domain.

As used herein, an “epitope” is a term in the art and refers to a localized region of an antigen to which an antibody can specifically bind. A region or a polypeptide con-tributing to an epitope can be contiguous amino acids of the polypeptide or an epitope can come together from two or more non-contiguous regions of the polypeptide.

As used herein, the term “heavy chain” when used in reference to an antibody refers to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4. In a specific embodiment, the heavy chain is a human heavy chain.

As used herein, the terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one of ordinary skill in the art. For example, a molecule that specifically binds to an antigen may bind to other peptides or polypeptides, generally with lower affinity as determined by, e.g., immunoassays, Biacore™, KinExA 3000 instrument (Sapidyne Instruments, Boise, ID), or other assays known in the art. In a specific embodiment, molecules that immunospecifically bind to an antigen bind to the antigen with a Ka that is at least 2 logs, 2.5 logs, 3 logs, 4 logs or greater than the Ka when the molecules bind to another antigen. In another specific embodiment, molecules that immunospecifically bind to an antigen do not cross react with other proteins. In another specific embodiment, molecules that immunospecifically bind to an antigen do not cross react with other non-KIT proteins.

As used herein, an “isolated” or “purified” antibody is substantially free of cellular materials or other contaminating proteins from the cell or tissue source from which the antibody is derived, or substantially free of chemical precursors or other chemicals when chemically synthesized.

As used herein, the term “light chain” when used in reference to an antibody refers to any distinct types, e.g., kappa (κ) of lambda (λ) based on the amino acid sequence of the constant domains. Light chain amino acid sequences are well known in the art. In specific embodiments, the light chain is a human light chain.

As used herein, the term “monoclonal antibody” refers to an antibody obtained from a population of homogenous or substantially homogeneous antibodies, and each monoclonal antibody will typically recognize a single epitope on the antigen. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single hybridoma or other cell (e.g., host cell producing a recombinant antibody), wherein the antibody immunospecifically binds to a c-Kit epitope (e.g., an epitope of a D2 and/or D3 of human c-Kit) as determined by, e.g., ELISA or other antigen-binding or competitive binding assay known in the art or in the Examples provided herein. Monoclonal antibodies described herein can, for example, be made by the hybridoma method as described in Kohler et al.; Nature, 256:495 (1975) or can be isolated from phage libraries using the techniques as described herein, for example. Other methods for the preparation of clonal cell lines and of monoclonal antibodies expressed thereby are well known in the art (see, e.g., Chapter 11 in: Short Protocols in Molecular Biology, (2002) 5th Ed., Ausubel et al., eds., John Wiley and Sons, New York). In specific embodiments, a monoclonal antibody is a monospecific antibody in that its antigen binding regions are specific for the same epitope. In further specific embodiments, a monoclonal monospecific antibody can be monovalent (having one antigen binding region) or multivalent (having more than one antigen binding regions), for example, bivalent (having two antigen binding regions).

As used herein, the term “naked antibody” refers to an antibody which is not linked, fused or conjugated to another agent or molecule (e.g., label or drug), peptide or polypeptide. In specific embodiments, a naked antibody expressed by a mammalian host cell can be glycosylated by the host cell's glycosylation machinery, for example glycosylation enzymes. In certain embodiments, a naked antibody is not glycosylated when it is expressed by a host cell which does not have its own glycosylation machinery, for example glycosylation enzymes. In certain embodiments, a naked antibody is a whole antibody, and in other embodiments, a naked antibody is an antigen binding fragment of a whole antibody, such as a Fab antibody.

As used herein, the term “polyclonal antibodies” refers to an antibody population that includes a variety of different antibodies directed to the same and to different epitopes within an antigen or antigens. Methods for producing polyclonal antibodies are known in the art (See, e.g., see, for example, Chapter 11 in: Short Protocols in Molecular Biology, (2002) 5th Ed., Ausubel et al., eds., John Wiley and Sons, New York).

As used herein, the term “recombinant human antibody” includes human antibodies that are isolated, prepared, expressed, or created by recombinant means, such as antibodies expressed using a recombinant expression vector transfected into a host cell, antibodies isolated from a recombinant, combinatorial human antibody library, antibodies isolated from an animal (e.g., a mouse, rabbit, goat, or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves creation, e.g., via synthesis, genetic en-gineering of DNA sequences that encode human immunoglobulin sequences, or splicing of sequences that encode human immunoglobulins, e.g., human immunoglobulin gene sequences, to other such sequences. Such recombinant human antibodies can have variable and constant regions derived from human germline immunoglobulin sequences. In certain embodiments, the amino acid sequences of such recombinant human antibodies have been modified such thus the amino acid sequences of the heavy chain variable region (“VH”) and/or light chain variable region (“VL”) of the recombinant antibodies are sequences that, while derived from and related to human germline VH and VL sequences, do not naturally exist within the human antibody germline repertoire in vivo. As a non-limiting example, a recombinant human antibody can be obtained by assembling several human sequence fragments into a composite human sequence of a recombinant human antibody.

As used herein, the terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain, which differ extensively in sequence among antibodies and are used in the binding and specificity of a particular antibody for its particular antigen. The variability in sequence is concentrated in those regions called comple-mentarity determining regions (CDRs) while the more highly conserved regions in the variable domain are called framework regions (FRs). Without wishing to be bound by any particular mechanism or theory, it is believed that the CDRs of the light and heavy chains are primarily responsible for the interaction of the antibody with an antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs). As a non-limiting example, a variable region described herein is obtained from assembling two or more fragments of human sequences into a composite human sequence.

As used herein, the term “cross-compete” or “cross-competition” means the antigen binding proteins compete for the same epitope or binding site on a target. Such competition can be determined by an assay in which the reference antigen binding protein (e.g., antibody or antigen-binding portion thereof) prevents or inhibits specific binding of a test antigen binding protein, and vice versa. Numerous types of competitive binding assays can be used to determine if a test molecule competes with a reference molecule for binding. Examples of assays that can be employed include solid phase direct or indirect radioimmunoassay (RIA), solid phase direct or indirect enzyme immunoassay (EIA), sandwich competition assay (see, e.g., Stahli et al. (1983) Methods in Enzymology 9:242-253), solid phase direct biotin-avidin EIA (see, e.g., Kirkland et al. (1986) J. Immunol. 137:3614-3619), solid phase direct labeled assay, solid phase direct labeled sandwich assay, Luminex (Jia et al., “A novel method of Multiplexed Competitive Antibody Binning for the characterization of monoclonal antibodies” J. Immunol. Methods (2004) 288, 91-98) and surface plasmon resonance (SPR) (Song et al., “Epitope Mapping of Ibalizumab, a Humanized Anti-CD4 Monoclonal Antibody with Anti-HIV-1 Activity in Infected Patients” J. Virol. (2010) 84, 6935-6942). Usually, when a competing antigen binding protein (or anti-c-Kit antibody or antigen binding fragment thereof) is present in excess, it will inhibit binding of a reference antigen binding protein (or a reference antibody) to a common antigen by at least 50%, 55%, 60%, 65%, 70%, or 75%. In some instances, binding is inhibited by at least 80%, 85%, 90%, 95%, 96%, 97%, 98%, 99%, or more.

As used herein, the term “reference antibody” means an antibody that serves as a basis of comparison for the analysis of a competing antibody that binds to the same epitope, an overlapping epitope, or an adjacent epitope, and cross-competes with said competing antibody for binding to an epitope.

In the present disclosure, the term “pharmaceutical composition” may refer to a form of a pharmaceutical preparation that allows the biological activity of an active ingredient contained in the composition to be effective. In one embodiment, the pharmaceutical composition is a composition comprising one or more active ingredients that can be administered to a patient and does not further comprise an unacceptable toxic component.

In the present disclosure, the term “pharmaceutically acceptable” may refer to a property of substance that is useful for the preparation of generally safe, non-toxic and biologically or otherwise desirable pharmaceutical compositions and is acceptable for human pharmaceutical applications and/or veterinary applications. In one embodiment, the term “pharmaceutically acceptable” means approved or approvable by a government regulatory agency or listed in U.S. Pharmacopeia, European Pharmacopeia or other generally recognized pharmacopoeia for use in humans and/or animals.

In the present disclosure, the term “pharmaceutically acceptable excipient” refers to any component that is not therapeutically active (inert) and non-toxic. In one embodiment, the pharmaceutically acceptable excipient may include, but is not limited to, for example, a binder, filler, solvent, buffer, tonicity agent, stabilizer, antioxidant, surfactant or lubricant configured to be used in formulating a pharmaceutical product.

In the present disclosure, the term “pharmaceutically acceptable carrier” refers to a component of a pharmaceutical composition other than an active ingredient, the component being non-toxic to a subject. In one embodiment, the pharmacologically acceptable carrier may include, but is not limited to, for example, a buffer, excipient, stabilizer or preservative.

In the present disclosure, the terms “pharmaceutically effective dose,” “pharmaceutically effective amount,” “administration dose,” “administration amount,” “therapeutically effective dose,” “therapeutically effective amount,” “effective dosage” or “effective amount” of a pharmaceutical composition may refer to an amount of a pharmaceutical composition (to be administered for the required duration) that is sufficient to achieve a therapeutic response or effect, a desired local or systemic therapeutic result or a desired preventive result. In one embodiment, the above terms refer to an amount of a pharmaceutical composition that, when administered to a subject, (i) treats or prevents, (ii) mitigates, reduces, ceases, attenuates, ameliorates or eliminates one or more symptoms of, or (iii) prevents or delays the onset of one or more symptoms of the disease, condition or disorder described herein (e.g., mast cell related disorder).

In the present disclosure, the terms “subject” or “patient” refers to any mammal. A subject or a patient described as “in need thereof” refers to one in need of a treatment (or prevention) for a disease. Mammals (i.e., mammalian animals) include humans, laboratory animals (e.g., primates, rats, mice), livestock (e.g., cows, sheep, goats, pigs), and household pets (e.g., dogs, cats, rodents). The subject may be a human. The subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. In some embodiments, the subject is a human. The subject may be healthy, or may be suffering from a mast cell related disorder at any developmental stage, wherein any of the stages are either caused by proliferation or accumulation of mast cell, or may be at risk of developing mast cell related disorders. In some embodiments, a subject has mast cell related disorders. In some embodiments, a subject has mast cell related disorders that are selected from the group consisting of chronic spontaneous urticaria, cold induced urticaria, symptomatic dermatographism and normo-complementemic urticarial vasculitis.

In the present disclosure, the term “treating” a disease in a subject or “treating” a subject having or suspected of having a disease refers to subjecting the subject to a pharmaceutical treatment, e.g., the administration of one or more agents, such that at least one symptom of the disease is decreased or prevented from worsening. Thus, in one embodiment, “treating” refers, inter alia, to delaying progression, expediting remission, inducing remission, augmenting remission, speeding recovery, increasing efficacy of or decreasing resistance to alternative therapeutics, or a combination thereof.

In the present disclosure, “administration” broadly refers to a route of administration of a composition to a subject. Examples of routes of administration include oral administration, rectal administration, topical administration, inhalation (nasal) or injection. Administration by injection includes intravenous (IV), intramuscular (IM), and subcutaneous (SC) administration. The pharmaceutical compositions described herein can be administered in any form by any effective route, including but not limited to oral, parenteral, enteral, intravenous, intraperitoneal, topical, transdermal (e.g, using any standard patch), intradermal, (intra) nasally, local, non-oral, such as aerosol, inhalation, subcutaneous, intramuscular, (trans) rectal, vaginal, intra-arterial, and intrathecal, transmucosal (e.g., (trans) urethral, vaginal (e.g., trans- and perivaginally), implanted, intravesical, intrapulmonary, intraduodenal, intragastrical, and intrabronchial. In certain embodiments, the pharmaceutical compositions described herein are administered orally, rectally, topically, intravesically, by injection into or adjacent to a draining lymph node, intravenously, by inhalation or aerosol, or subcutaneously. In some certain embodiments, the pharmaceutical compositions described herein are administered intravenously.

In the present disclosure, the term “c-Kit” refers to a type III receptor tyrosine kinase which is also known as receptors of stem cell factor (SCF). “c-Kit” includes any variant or isoform of c-Kit naturally expressed by cells. Thus, the anti-c-Kit antibodies disclosed herein may cross-react with different isoforms of the same species (e.g., different isoforms of human c-Kit), or may cross-react with c-Kit of a species other than human (e.g., mouse c-Kit). Alternatively, anti-c-Kit antibodies may be specific to human c-Kit and may not cross-react with c-Kit of other species. The c-Kit, or any variants and isoforms thereof, may be separated from cells or tissues that naturally express them or recombinantly generated. In one embodiment, the sequence (Q26 to D309) of Domains 1 to 3 of human c-Kit, excluding 25 signal peptides, may be SEQ ID NO: 14.

### II. Anti-c-Kit Antibody

Provided herein are anti-c-Kit antibodies that specifically bind to a human c-Kit (e.g., extracellular domain or Ig-like domain of a human c-Kit receptor), or an antigen binding fragment thereof, for use in methods for preventing, treating or managing mast cell related disorders. Suitable anti-c-Kit antibodies for use in the methods provided herein can be selected as described herein.

c-Kit has five immunoglobulin-like domains (D1-D5) in the extracellular compartment and two kinase domains in the intracellular compartment. Stem cell factor (SCF), a ligand of c-Kit, binds to D1-D3 (See, e.g., Liu H, et al., EMBO J 26:891-901). SCF binding induces the homodimerization of c-Kit, which leads to phosphorylation. Phospho-c-Kit activates multiple signaling pathways, including phosphatidylinositol-3-kinase (PI3K)-Akt, mitogen-activated protein kinase (MAPK)-ERK, SRC, Janus kinase (JAK)-signal transducer and activator of transcription (STAT) pathways, resulting in cell proliferation, survival, differentiation, and migration (See, e.g., Lennartsson J et al., (2012) Physiol Rev 92:1619-49). SCF/c-Kit is essential for the growth, survival, and differentiation in mast cells, and also induces migration and invasion into specific tissues through SCF-chemotaxis (See, e.g., Cruse G et al (2014), Immunol Allergy Clin North Am 34:219-37; El-Agamy D S (2012), Eur J Pharmacol 690:1-3; and Hundley T R et al (2004), Blood 104:2410-7). Furthermore, SCF enhances the inflammatory response by synergistically increasing the degranulation and production of cytokines (See, e.g., Okayama Y et al (2006), Immunol Res 34:97-115; and Varricchi G et al (2018), Immunol Rev 282:8-34). In the present disclosure, an antibody that binds to Ig-like domains D2 and/or D3 of c-Kit and inhibits the binding of SCF to c-Kit was found to inhibit the SCF/c-Kit signaling and thus suppress or inhibit the phosphorylation of c-Kit and proliferation, migration and/or degranulation of mast cells. In one embodiment, the antibody in the present disclosure may specifically bind to c-Kit with certain equilibrium dissociation constant (KD) value. In one embodiment, the antibody in the present disclosure may specifically bind to c-Kit with a binding affinity which is greater than that between SCF and c-Kit. The antibody in the present disclosure, with one or more characteristics described above, may suppress or inhibit c-Kit phosphorylation and proliferation, migration and/or degranulation of mast cells and thereby treat, prevent, manage, and/or ameliorate one or more mast cell related disorders.

In one embodiment, the anti-c-Kit antibody of the present disclosure or the antigen binding fragment thereof may inhibit the binding between c-Kit and SCF in mast cells and thereby inhibit or suppress SCF/c-Kit signaling. In one embodiment, the anti-c-Kit antibody of the present disclosure or the antigen binding fragment thereof may immunospecifically bind to certain Ig-like domain(s) of human c-Kit. Specifically, in one embodiment, the anti-c-Kit antibody of the present disclosure or the antigen binding fragment thereof may immunospecifically bind to Ig-like domain D2 and/or D3 of human c-Kit, and further specifically certain epitope of Ig-like domain D2 and D3, as explained below. By such binding described above, the anti-c-Kit antibody of the present disclosure or the antigen binding fragment thereof may inhibit SCF/c-Kit signaling; suppress proliferation, migration, and degranulation in mast cells; and/or suppress the secretion of pro-inflammatory cytokines which can exacerbate allergic symptoms of mast cell related disorders. In one embodiment, an anti-c-Kit antibody or an antigen binding fragment thereof may be defined as an antibody that targets certain epitope of c-Kit (e.g., Ig-like domain D2 and/or D3 as further described below), and any antibody that immunospecifically binds to such epitope may be used as and included in the scope of, without limitation, the anti-c-Kit antibody of the present disclosure.

In certain embodiments, the anti-c-Kit antibody of the present disclosure or an antigen binding fragment thereof may specifically bind to the Ig-like domains D1-D3 of human c-Kit (SEQ ID NO: 14), to Ig-like domain D2 and D3 of human c-Kit (SEQ ID NO: 13), to R112-R205 region of Ig-like domain D2 of human c-Kit (SEQ ID NO: 11) and S240-H263 region of Ig-like domain D3 of human c-Kit (SEQ ID NO: 12), or to epitope of R122, Y125, R181, K203, R205, S261, and H263 of Ig-like domain D2 and D3 of human c-Kit.

In one embodiment, the anti-c-Kit antibody or the antigen binding fragment thereof may specifically bind to at least one of R122, Y125, R181, K203, R205, S261 and H263 on amino acid sequence of SEQ ID NO: 14. In certain embodiments, the anti-c-Kit antibody or the antigen binding fragment thereof may specifically bind to R122, Y125, R181, K203, R205, S261 and H263 on amino acid sequence of SEQ ID NO: 14. In one embodiment, SEQ ID NO: 14 is the sequence of Ig-like domains D1 to D3 of human c-Kit (Q26 to D309), excluding 25 sequences of signal peptides, and the number of each amino acid above (e.g., R122) is the number of amino acid including 25 sequence of signal peptide. Thus, for example, R122 means the 97th amino acid sequence in SEQ ID NO: 14, and Y125 means the 100th amino acid sequence. The remaining amino acids can be interpreted in the same manner.

In certain embodiments, the anti-c-Kit antibody or the antigen binding fragment thereof may have equilibrium dissociation constant (KD) value for human c-Kit less than or equal to 10⁻¹¹ M. In one embodiments, the anti-c-Kit antibody or the antigen binding fragment thereof may have equilibrium dissociation constant (KD) value for human c-Kit less than or equal to 10⁻⁸ M, 10⁻⁹ M, 10⁻¹⁰ M, 10⁻¹¹ M, 8×10⁻¹² M, 6×10⁻¹² M, 4×10⁻¹² M, 3×10⁻¹² M, 2×10⁻¹² M, 1×10⁻¹² M, or 10⁻¹³ M. In one embodiment, the equilibrium dissociation constant (KD) value for human c-Kit of the anti-c-Kit antibody or the antigen binding fragment thereof may be measured by a known method in the field, including and not limited to, such, as surface plasmon resonance (SPR). Specifically, using, e.g., SR7500DC (Reichert, USA), human c-Kit used for antibody preparation may be fixed on a PEG (Reichert, USA) chip. Thereafter, after flowing the anti-c-kit antibody by concentration, the KD value, which is the affinity for c-Kit, may be analyzed using, e.g., the Scrubber2 program. In certain embodiments, the anti-c-Kit antibody of the present disclosure may bind to c-Kit with the above KD, thereby inhibiting the binding between SCF and c-Kit or showing effect such as inhibiting c-Kit phosphorylation or treating, preventing, managing, and/or ameliorating one or more mast cell related disorders. In one embodiment, the antibody or the antigen binding fragment thereof specifically binds to c-Kit with binding affinity which is more than 50-fold stronger, 100-fold stronger, 150-fold stronger, 200-fold stronger, 250-fold stronger, 300-fold stronger, 350-fold stronger, 400-fold stronger, 450-fold stronger, 500-fold stronger, 550-fold stronger, or 600-fold stronger than that between SCF and c-Kit. In certain embodiments, the anti-c-Kit antibody of the present disclosure may bind to c-Kit with the above KD, thereby inhibiting the binding between SCF and c-Kit or showing effect such as inhibiting c-Kit phosphorylation or treating, preventing, managing, and/or ameliorating one or more mast cell related disorders.

Antigen binding proteins that have an identical epitope or overlapping epitope will often cross-compete for binding to the antigen. Thus, in certain embodiments, the anti-c-Kit antibody of the present disclosure may cross-compete for binding to human c-Kit with a reference antibody.

In one embodiment, the anti-c-Kit antibody or the antigen binding fragment thereof may cross-compete for binding to human c-Kit epitope on an amino acid sequence of SEQ ID Nos: 11 and/or 12 with a reference antibody comprising VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5, and/or VL CDR3 of SEQ ID NO: 6.

In one embodiment, the anti-c-Kit antibody or the antigen binding fragment thereof may cross-compete for binding to human c-Kit epitope on an amino acid sequence of SEQ ID Nos: 11 and/or 12 with a reference antibody comprising a VH comprising SEQ ID NO: 7 and/or a VL comprising SEQ ID NO: 8.

In one embodiment, the anti-c-Kit antibody or the antigen binding fragment thereof may comprise VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5 and/or VL CDR3 of SEQ ID NO: 6.

In one embodiment, the anti-c-Kit antibody or the antigen binding fragment thereof may comprise a VH comprising SEQ ID NO: 7 and/or a VL comprising SEQ ID NO: 8.

In one embodiment, the anti-c-Kit antibody or the antigen binding fragment thereof, not limited thereto, may comprise modification, deletion or substitution of one or two amino acid(s) present in CDR.

In one embodiment, the anti-c-Kit antibody or the antigen binding fragment thereof comprises a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 7 and/or a VL comprising an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 8.

In one embodiment, the anti-c-Kit antibody or the antigen binding fragment thereof may comprise a heavy chain comprising SEQ ID NO: 9 and a light chain comprising SEQ ID NO: 10.

In one embodiment, antibodies may include, for example, any one or more of monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecule, an antibody light chain monomer, an antibody heavy chain monomer, an antibody light chain dimer, an antibody heavy chain dimer, an antibody light chain-antibody heavy chain pair, in-trabodies, heteroconjugate antibodies, single domain antibodies, monovalent antibodies, single chain antibodies or single-chain Fvs (scFv), camelized antibodies, affybodies, Fab fragments, F(ab′) fragments, disulfide-linked Fvs (sdFv), anti-idiotypic (anti-Id) antibodies (including, e.g., anti-anti-Id antibodies), and epitope-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof.

In one embodiment, the anti-c-Kit antibody of the present disclosure may be further modified to reduce effector functions, which cause complement-dependent cytotoxicity (CDC) and/or antibody-dependent cellular cytotoxicity (ADCC). Antibody drug administration can cause hypersensitivity reactions (HSRs). In particular, therapeutic antibodies may induce mast cell degranulation via Fc gamma receptor (FcγRs), which are upregulated in human mast cells, and cross-linking of FcγRs by IgG induces degranulation, similar to FcεRI (See, e.g., Caslin H L et al (2018), etc.). LOP628, an anti-c-Kit antibody-drug conjugate developed by Novartis, failed in a phase I clinical trial due to HSR. This is because the immune complex formed from c-Kit-LOP628 increased FcγRI-mediated degranulation of human mast cells. It is reported that the large immune complex composed of IgG and antigen binds to FcγRs with high avidity and can induce FcγR cross-linking, leading to degranulation (See, e.g., Bournazos S et al (2020), Nat Rev Immunol 20:633-643). In addition, given that IFN-γ enhances expression of FcγRI and FcγRI-mediated degranulation, it could be particularly fatal in allergic patients (L'Italien L et al (2018), etc.). Further, the excessive effector function of a therapeutic antibody could be induced by galactosylation and afucosylation of IgG. It has been known that a high degree of galactosylation can increase complement activation by enhancing C1q binding and CDC, and can have modest effects on ADCC (D. Reusch et al, Glycobiology 25 (12) (2015) 1325-1334). In addition, a decrease in core-fucose levels results in a significant increase in ADCC due to an increased affinity of IgG1 for FcγRIIIa (X. R. Jiang et al, Nat. Rev. Drug Discov. 10 (2) (2011) 101-111). Furthermore, the lack of fucose promotes antibody-dependent cellular phagocytosis mediated by FcγRIIIa-positive monocytes and macrophages (S. Herter et al, J. Immunol. 192 (5) (2014) 2252-2260). On the other hand, the increased GOF of IgG reduces its binding affinity with FcγRs, thereby reducing ADCC (G. P. Subedi et al, MAbs 8 (8) (2016) 1512-1524). Therefore, galactosylation should be decreased and fucosylation of antibodies should be increased to reduce unwanted hypersensitivity reactions. In one embodiment, the anti-c-Kit antibody of the present disclosure can be modified to have no effector function or reduce effector function by lowering its affinity with FcγRs, and specifically, the Fc region of the anti-c-Kit antibody of the present disclosure may be modified to remove or reduce its effector function. In one embodiment, the anti-c-Kit antibody of the present disclosure can be manufactured or expressed by using Chinese Hamster Ovary (CHO) cell as transfected cells or host cells.

In one embodiment, the anti-c-Kit antibody of the present invention may be 2G4 antibody, but not limited thereto. 2G4 antibody is a fully human anti-c-Kit antibody and binds to the Ig-like domain D2 and/or D3 of c-Kit and blocks SCF binding, thereby showing c-Kit inhibitory effect. The c-Kit binding affinity, epitope map, c-Kit-positive cell-binding ability, effector function, immunogenicity, and crystal structure of the highly fucosylated form of 2G4 was described in Kim J O, et al., (2020) Int J Biol Macromol 159:66-78 and used in the Examples described below.

### III. Treatment

In one embodiment, provided herein are pharmaceutical compositions for treating, preventing or ameliorating mast cell related disorders comprising one or more anti-c-Kit antibodies provided herein, or antigen-binding fragments thereof.

In one embodiment, provided herein are compositions, such as pharmaceutical compositions, comprising one or more anti-c-Kit antibodies (e.g., humanized antibodies) for use in the methods described herein, or antigen-binding fragments thereof. In particular embodiments, compositions described herein can be for in vitro, in vivo, or ex vivo uses. In specific embodiments, provided herein is a pharmaceutical composition comprising an anti-c-Kit antibody (e.g., a humanized antibody) for use in the methods described herein (or an antigen-binding fragment thereof) and a pharmaceutically acceptable carrier or excipient.

In one embodiment, provided herein are methods of treating, preventing or ameliorating mast cell related disorders in a subject, comprising administering to a subject in need thereof one or more anti-c-Kit antibodies provided herein, or antigen-binding fragments thereof. A subject in need thereof may include, for example, a subject who has been diagnosed with mast cell related disorders, or a subject who has been treated, including subjects that have been refractory to the previous treatment.

In one embodiment, provided herein are the use of anti-c-Kit antibodies provided herein or antigen-binding fragments thereof for the treatment, prevention or amelioration of mast cell related disorders.

In one embodiment, provided herein are anti-c-Kit antibodies provided herein or antigen-binding fragments thereof for use in a method for treating, preventing or ameliorating mast cell related disorders, or a pharmaceutical composition comprising the same.

In certain embodiments, the methods provided herein include administering the anti-c-Kit antibody in conjunction with a second therapeutic agent to the subject. Con-junctive therapy includes sequential, simultaneous and separate, or co-administration of the active compound in a way that the therapeutic effects of the first agent administered have not entirely disappeared when the subsequent agent is administered. In certain embodiments, the second agent may be co-formulated with the first agent or be formulated in a separate pharmaceutical composition.

### Mast Cell Related Disorders

The main cause of mast cell related disorders includes excessive proliferation and abnormal activation of mast cells. Thus, inhibition of proliferation or migration of mast cells may result in the treatment of mast cell related disorders by reducing the number of mast cells in organs or tissues. In one embodiment, the anti-c-Kit antibody of the present disclosure or the antigen-binding fragment inhibits or suppresses SCF-mediated c-Kit phosphorylation, proliferation or migration of mast cells, and thus can be used as a therapeutic agent for treating, preventing, ameliorating, or managing mast cell related disorders and/or one or more symptoms thereof.

Allergic diseases, including and not limited to, e.g., asthma, chronic spontaneous urticaria (CSU), allergic rhinitis (AR), atopic dermatitis (AD), food allergies, and anaphylaxis, are the most common mast cell related disorders. For example, it has been found that the number of mast cells and the level of inflammatory mediators increase in the bronchi of asthma patients. An increase in the number of mast cells in the airway induces increased inflammation and immune remodeling. The immune remodeling results in the recruitment of various inflammatory cells, such as eosinophils, basophils, and helper T cells, leading to further exacerbation of the symptoms of mast cell related disorders (See, e.g., Thomson N C et al (2012), Clin Med Insights Circ Respir Pulm Med 6:27-40). Asthma is an allergic disease that occurs in the lungs and airways due to an inordinate inflammatory response triggered by mast cells (See, e.g., Brown J M et al (2008)). The level of SCF significantly increases in asthmatic airways, leading to mast cell recruitment, proliferation, and survival (See, e.g., Al-Muhsen S Z et al (2004), Clin Exp Allergy 34:911-6). In a previous study, it was demonstrated that SCF increased airway hyperreactivity, which was inhibited by SCF neutralization (See, e.g., Campbell E et al (1999), Am J Pathol 154:1259-65). Thus, the SCF/c-Kit signaling in mast cells closely contributes to asthma and the anti-c-Kit antibody of the present disclosure which can inhibit SCF/c-Kit signaling in mast cells can be used as a therapeutic agent for treating, preventing, managing or ameliorating asthma and/or one or more symptoms thereof.

In CSU, AR, and AD, the accumulation of mast cells and increased levels of inflammatory mediators released by mast cells are the main causes of these diseases (See, e.g., Min T K et al (2019), Allergy Asthma Immunol Res 11:470-481). Thus, the anti-c-Kit antibody of the present disclosure which can inhibit or suppress proliferation or migration of mast cells and inflammatory mediators (e.g., cytokines released by mast cells) can be used as a therapeutic agent for treating, preventing, managing or ameliorating CSU, AR, and AD, and/or one or more symptoms thereof.

In mast cell activation disease (MCAD) such as mast cell leukemia (MCL), increase in the number of mast cells has been observed (Haenisch B et al (2012), Immunology 137:197-205), and the major cause of MCAD or MCL is an excessive increase in the number of, or abnormal activation of, mast cells. Thus, the anti-c-Kit antibody of the present disclosure which can inhibit or suppress proliferation or migration of mast cells and inflammatory mediators (e.g., cytokines released by mast cells) can be used as a therapeutic agent for treating, preventing, managing or ameliorating MCAD (e.g., MCL), and/or one or more symptoms thereof.

In one embodiment, mast cell related disorders may include and are not limited to mastocytosis and mast cell activation syndrome (MCAS). The major cause of mastocytosis is the expansion and accumulation of abnormal (neoplastic) mast cells in the skin (cutaneous) or other organs (systemic); it manifests various symptoms, such as itching, hives, vascular instability, headache, enlarged liver, and anaphylactic shock (See, e.g., Nedoszytko B et al (2021), Int J Mol Sci 22). Given that multifocal dense in-filtrates of mast cells (>15 mast cells in aggregates) in bone marrow or other tissue (commonly skin) biopsies is one of the major criteria in the diagnosis of mastocytosis, it is evident that accumulation of mast cells is a major cause of mastocytosis (See, e.g., Akin C (2017), J Allergy Clin Immunol 140:349-355). These abnormal mast cells mainly carry gain-of-function mutations of c-Kit, and c-Kit mutations were found in about 90% of patients with mastocytosis (See, e.g., Chatterjee A et al (2015), On-cotarget 6:18250-64). The causes and symptoms of MCAS are similar to those of systemic mastocytosis, but mast cells do not accumulate in specific organs (See, e.g., Frieri M et al (2013), Curr Allergy Asthma Rep 13:27-32). MCAS may be characterized by symptoms appearing in more than one organ, and symptoms appearing cyclically. Thus, the anti-c-Kit antibody of the present disclosure which can inhibit or suppress proliferation or migration of mast cells and inflammatory mediators (e.g., cytokines released by mast cells) can be used as a therapeutic agent for treating, preventing, managing or amerliorating mastocytosis and MCAS, and/or one or more symptoms thereof.

Mast cells secrete various cytokines that recruit and stimulate various immune cells. Therefore, the modulation of cytokine secretion by mast cells can augment the inflammatory response, and increased inflammatory response can be a serious factor that aggravates symptoms in allergy patients (See, e.g., Velez T E et al (2018), Curr Allergy Asthma Rep 18:30). Thus, if the cytokines secreted from mast cells can be controlled or managed, one or more associated inflammatory responses can be suppressed, and therefore one or more symptoms of mast cell related disorder patients or allergy patients can be ameliorated and/or managed.

It is known that GM-CSF is essential for the development, function, and survival of eosinophils. Increased GM-CSF levels in lesions of patients with asthma and CSU induce eosinophil recruitment and survival, resulting in excessive accumulation of eosinophils (See, e.g., Altrichter S et al (2020), J Allergy Clin Immunol 145:1510-1516). Eosinophils, as well as mast cells, are considered therapeutic targets for allergic diseases because they play a critical role in chronic and severe symp-tomatology (See, e.g., O'Sullivan J A et al (2020), J Leukoc Biol 108:73-81). Eosinophils produce and secrete fibrogenic factors, such as fibroblast growth factor (FGF), heparin binding epidermal growth factor, IL-4, IL-13, IL-17, nerve growth factor, platelet derived growth factor, and transforming growth factor-β (TGF-β), leading to the development of severe asthma (See, e.g., Aceves S S et al (2008), Curr Mol Med 8:350-8). Therefore, inhibition of GM-CSF secretion from mast cells by the anti-c-Kit antibody of the present disclosure may show a synergistic activity against mast cell related disorders or allergic diseases by suppressing eosinophil recruitment and survival. Further, c-Kit is also expressed in eosinophils, and it is reported that the activation of SCF/c-Kit signaling in eosinophils enhances the expression of FGF-5, FGF-7, and TGF-β (See, e.g., Dolgachev V et al (2008), Am J Pathol 172:68-76). Therefore, in addition to reducing the recruitment of eosinophils through mast cell in-activation, the anti-c-Kit antibody of the present disclosure may directly exhibit therapeutic effects by inhibiting the SCF/c-Kit signal in eosinophils.

Other cytokines whose secretion is increased by SCF in mast cells can also exacerbate allergic response in mast cell related disorders. VEGF promotes the migration of inflammatory cells by increasing vascular permeability, and CCL2 promotes the recruitment of various inflammatory cells through chemotaxis (See, e.g., Ribatti D et al (2012), Biochim Biophys Acta 1822:2-8). BDNF induces bronchoconstriction by promoting the proliferation of airway smooth muscle cells in asthmatic patients, and C5/C5a contributes to the pathological features of asthma, such as mucus release, con-traction of smooth muscle cells, increased vascular permeability, and infiltration of inflammatory cells (See, e.g., Khan M A et al (2014), Respir Med 108:543-9). Meanwhile, a few cytokines, including IL-2, which can cause an anti-inflammatory response, are reduced by SCF. IL-2 promotes the function and survival of regulatory T cells (Tregs), which play a role in preventing allergic diseases, such as allergic rhinitis (AR) and atopic dermatitis (AD), by regulating immune homeostasis (See, e.g., Noval Rivas M et al (2016), J Allergy Clin Immunol 138:639-652). The IL-2 downregulation induced by SCF reduces the activity of Treg, thereby increasing the inflammatory response.

As such, SCF can modulate cytokines secretion by mast cell and the modulation mainly shown by an increase in various pro-inflammatory cytokines and a decrease in a small number of anti-inflammatory cytokines. The anti-c-Kit antibody of the present disclosure exhibits the efficacy or effects of treating, preventing, ameliorating, and/or managing mast cell related disorders by inhibiting SCF-mediated cytokine modulation in mast cells, which accelerates the progression and symptoms of mas cell related disorder and/or one or more symptoms thereof.

In certain embodiments, the mast cell related disorders may include and are not limited to, mast cell activation syndrome (MCAS), primary mast cell activation syndrome, monoclonal mast cell activation syndrome (MMAS), idiopathic mast cell activation syndrome, secondary mast cell activation syndrome, asthma, allergic rhinitis, allergic inflammation, food allergies, rheumatoid arthritis, inflammatory bowel disease (IBD), multiple sclerosis, anaphylaxis, idiopathic anaphylaxis, Ig-E mediated anaphylaxis, non-Ig-E mediated anaphylaxis, neurofibromatosis, atopic dermatitis, psoriasis, urticaria, idiopathic urticaria, chronic urticaria, chronic spontaneous urticaria, allergic urticaria, idiopathic urticaria, cold induced urticaria, heat induced urticaria, dermatographic urticaria, vibratory urticaria, cholinergic urticaria, contact urticaria, symptomatic dermatographism, normo complementemic urticarial vasculitis, hypereosinophilic syndrome, fibrosis, idiopathic pulmonary fibrosis (IPF), pulmonary fibrosis, hepatic fibrosis, cardiac fibrosis, scleroderma, myelofibrosis, inflammatory conditions, pulmonary arterial hypertension (PAH), irritable bowel syndrome (IBS), dermatosis, mast cell activation disorder (MCAD), mastocytosis, cutaneous mastocytosis (e.g., urticaria pigmentosa), indolent systemic mastocytosis (ISM), smoldering systemic mastocytosis (SSM), mastocytosis with an associated hematologic neoplasm (SM-AHN), aggressive systemic mastocytosis (ASM), mast cell leukemia (MCL), mast cell sarcoma, systemic mastocytosis (SM), swelling (angioedema), primary pulmonary hypertension (PPH), mastocytic gastroenteritis, mastocytic colitis, pruritus, chronic pruritis, pruritis secondary to chronic kidney failure and heart, heart, vascular, intestinal, brain, kidney, liver, pancreas, muscle, bone and skin conditions associated with mast cells, autoimmune diseases, respiratory diseases, allergic diseases (including, for example. food allergies), allergic sinusitis, allergic contact dermatitis, erythema nodosum, erythema multiforme, cutaneous necrotizing venulitis and insect bite skin inflammation, bronchial asthma, inflammatory diseases, diabetes, type I diabetes, type II diabetes, central nervous system (CNS) disorders, interstitial cystitis and hematologic disorders.

In certain embodiments, the mast cell related disorders may be selected from the group consisting of chronic spontaneous urticaria, cold induced urticaria, symptomatic dermatographism and normo-complementemic urticarial vasculitis.

### Compositions

Therapeutic formulations containing one or more anti-c-Kit antibodies (e.g., humanized antibodies) provided herein can be prepared for storage by mixing the antibody having the desired degree of purity with optional physiologically acceptable carriers, excipients or stabilizers (Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md.), in the form of lyophilized formulations or aqueous solutions. Acceptable carriers, excipients, or stabilizers are nontoxic to recipients at the dosages and concentrations employed, and may include buffers such as phosphate, citrate, and other organic acids; and/or non-ionic surfactants such as TWEEN™, PLURONICS™ or polyethylene glycol (PEG).

In one embodiment, formulations, such as those described herein, can also contain more than one active compounds (for example, molecules, e.g., antibody or antibodies described herein) as necessary for the particular indication being treated. In certain embodiments, formulations comprise an anti-c-Kit antibody provided herein and one or more active compounds with complementary activities that do not adversely affect each other. Such molecules are suitably present in combination in amounts that are effective for the purpose intended.

In one embodiment, the formulations to be used for in vivo administration can be sterile. This is readily accomplished by filtration through, e.g., sterile filtration membranes.

In specific embodiments, the pharmaceutical compositions provided herein contain therapeutically effective amounts of one or more of the anti-c-Kit antibodies (e.g., humanized antibodies) provided herein, and optionally one or more additional prophylactic or therapeutic agents, in a pharmaceutically acceptable carrier. Such pharmaceutical compositions are useful in the prevention, treatment, management or amelioration of mast cell related disorders, or one or more of the symptoms thereof.

In addition, anti-c-Kit antibodies provided herein can be formulated as the sole pharmaceutically active ingredient in the composition or can be combined with other active ingredients (such as one or more other prophylactic or therapeutic agents).

Compositions can contain one or more anti-c-Kit antibodies provided herein. In one embodiment, the antibodies are formulated into suitable pharmaceutical preparations, such as solutions, suspensions, powders, or elixirs, in sterile solutions or suspensions for parenteral administration. In one embodiment, the antibodies are formulated into suitable pharmaceutical preparations, such as solutions, suspensions, tablets, dis-persible tablets, pills, capsules, powders, sustained release formulations or elixirs, for oral administration.

In such compositions, one or more anti-c-Kit antibodies provided herein is (are) mixed with a suitable pharmaceutical carrier. Concentrations of an antibody or antibodies in the compositions can, for example, be effective for delivery of an amount, upon administration, that treats, prevents, protects against, ameliorates or manages mast cell related disorders, and/or one or more symptoms thereof.

In one embodiment, the compositions are formulated for single dosage administration. To formulate a composition, the weight fraction of compound is dissolved, suspended, dispersed or otherwise mixed in a selected carrier at an effective concentration such that the treated disorders are relieved, prevented, or one or more symptoms are ameliorated.

In certain embodiments, an anti-c-Kit antibody (e.g., a humanized antibody) provided herein is included in the pharmaceutically acceptable carrier in an effective amount sufficient to exert a therapeutically useful effect in the absence of, or with minimal or negligible, undesirable side effects on the patient treated.

In one embodiment, a therapeutically effective dosage produces a serum concentration of antibody of from about 0.1 ng/ml to about 50-100 μg/ml. The pharmaceutical compositions, in another embodiment, provide a dosage of from about 0.001 mg to about 2000 mg of antibody per kilogram of body weight for administration over a period of time, e.g., every day, twice or thrice every week, once every week, every 2 weeks, or every 3 weeks. Pharmaceutical dosage unit forms can be prepared to provide from about 0.01 mg to about 2000 mg, and in one embodiment from about 10 mg to about 500 mg of the antibody and/or a combination of other optional essential ingredients per dosage unit form.

An anti-c-Kit antibody described herein can be administered at once, or can be divided into a number of smaller doses to be administered at intervals of time.

Upon mixing or addition of an antibody, the resulting mixture can be a solution, suspension, emulsion or the like. The form of the resulting mixture depends upon a number of factors, including the intended mode of administration and the solubility of the anti-c-Kit antibody in the selected carrier or vehicle.

Pharmaceutical compositions described herein are provided for administration to humans and animals, such as mammals (e.g., cat or dog), in unit dosage forms, such as sterile parenteral (e.g., intravenous) solutions or suspensions containing suitable quantities of the ant-c-Kit antibodies or pharmaceutically acceptable derivatives thereof. Pharmaceutical compositions are also provided for administration to humans and animals, such as mammals (e.g., cat or dog), in unit dosage form, such as tablets, capsules, pills, powders, granules, and oral solutions or suspensions, and oil-water emulsions containing suitable quantities of the anti-c-Kit antibodies or pharmaceutically acceptable derivatives thereof. The anti-c-Kit antibody is, in one embodiment, formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the antibody sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules, or bottles of pints or gallons. Hence, multiple dose form is a multiple of unit-doses which are not segregated in packaging.

In certain embodiments, one or more anti-c-Kit antibodies described herein may be in a liquid pharmaceutical formulation. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md.

Dosage forms or compositions containing antibody in the range of 0.005% to 100% with the balance made up from non-toxic carrier can be prepared. Methods for preparation of these compositions are known to those skilled in the art.

Parenteral administration, in one embodiment, is characterized by injection, either subcutaneously, intramuscularly or intravenously is also contemplated herein. Injectables can be prepared in conventional forms, either as liquid solutions or suspensions, as solid forms suitable for solution or suspension in liquid prior to injection, or as emulsions. The injectables, solutions, and emulsions also contain one or more excipients. Suitable excipients may include and are not limited to, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents. Other routes of administration may include, epidural administration, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products (e.g., lyophilized powders), ready to be combined with a solvent just prior to use, hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers may include and are not limited to physiological saline or phosphate buffered saline (PBS), and/or solutions containing thickening and solubilizing agents, such as glucose, polyethylene glycol, and polypropylene glycol and any mixtures thereof.

Pharmaceutically acceptable carriers used in parenteral preparations may include and are not limited to aqueous vehicles, nonaqueous vehicles, antimicrobial agents, isotonic agents, buffers, antioxidants, local anesthetics, suspending and dispersing agents, emulsifying agents, sequestering or chelating agents, and/or other pharmaceutically acceptable substances.

Pharmaceutical carriers may also include ethyl alcohol, polyethylene glycol, and/or propylene glycol for water miscible vehicles; and sodium hydroxide, hydrochloric acid, citric acid, and/or lactic acid for pH adjustment.

In one embodiment, intravenous or intraarterial infusion of a sterile aqueous solution containing an active compound is an effective mode of administration. In another embodiment, a sterile aqueous or oily solution or suspension containing an active material may be injected to a subject as necessary to produce the desired pharmacological effect.

An anti-c-Kit antibody described herein can be suspended in micronized or other suitable form. The form of the resulting mixture depends upon a number of factors, including the intended mode of administration and the solubility of the anti-c-Kit antibody in the selected carrier or vehicle.

In other embodiments, the pharmaceutical formulations are lyophilized powders, which can be reconstituted for administration as solutions, emulsions, and/or other mixtures. They can also be reconstituted and formulated as solids or gels.

The lyophilized powder is prepared by dissolving an anti-c-Kit antibody provided herein, in a suitable solvent. In some embodiments, the lyophilized powder is sterile. The solvent can contain an excipient which improves the stability and/or other pharmacological components of the powder or reconstituted solution, prepared from the powder. Excipients that can be used include, but are not limited to, dextrose, sorbital, fructose, corn syrup, xylitol, glycerin, glucose, sucrose or other suitable agent. The solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the anti-c-Kit antibody. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound.

Antibodies described herein can be formulated for local or topical application, such as for topical application to the skin and mucous membranes, in the form of gels, creams, and lotions and for intracisternal or intraspinal application. Topical administration is contemplated for transdermal delivery and also for administration to the mucosa, or for inhalation therapies. Nasal solutions of the active compound alone or in combination with other pharmaceutically acceptable excipients can also be administered.

The antibodies and other compositions provided herein can also be formulated to be targeted to a particular tissue, receptor, or other areas of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In some embodiments, the anti-c-Kit antibodies described herein are targeted (or otherwise administered) to the bone marrow, the gas-trointestinal tract or the brain. In specific embodiments, an anti-c-Kit antibody described herein is capable of crossing the blood-brain barrier.

In particular embodiments, a composition comprising anti-c-Kit antibodies described herein can be targeted to the ear.

### Dosages and Administration

The dosage and frequency of administration of an anti-c-Kit antibody described herein or a pharmaceutical composition thereof to a subject in need thereof (e.g., mammal, such as human, dog or cat) are determined in accordance with the methods for treating mast cell related disorders provided herein such that the administration of an anti-c-Kit antibody described herein or a pharmaceutical composition thereof is ef-ficacious while minimizing side effects. The exact dosage of an anti-c-Kit antibody described herein to be administered to a particular subject or a pharmaceutical composition thereof can be determined in light of factors related to the subject that requires treatment. In one embodiment, factors which can be taken into account may include and are not limited to the severity of the disease state, general health of the subject, age, and weight of the subject, diet, time and frequency of administration, com-bination(s) with other therapeutic agents or drugs, reaction sensitivities, and tolerance/response to therapy. The dosage and frequency of administration of an anti-c-Kit antibody described herein or a pharmaceutical composition thereof can be adjusted over time to provide sufficient levels of the anti-c-Kit antibody or to maintain the desired effect.

The precise dose to be employed in the formulation will also depend on the route of administration, and the seriousness of mast cell related disorders, and may be decided according to the judgment of the practitioner and/or each patient's circumstances.

In certain embodiments, for the anti-c-Kit antibodies described herein, the dosage administered to a patient, to prevent, protect against, manage, or treat mast cell related disorders is typically 0.1 mg/kg to 100 mg/kg of the patient's body weight. Generally, human antibodies have a longer half-life within the human body than antibodies from other species due to the immune response to the foreign polypeptides. Thus, lower dosages of human antibodies and/or less frequent administration is often possible. Further, the dosage and frequency of administration of the antibodies described herein can be reduced by enhancing uptake and tissue penetration of the antibodies by modifications such as, for example, lipidation.

In one embodiment, approximately 0.001 mg/kg (mg of antibody per kg weight of a subject) to approximately 500 mg/kg of an anti-c-Kit antibody described herein is administered to prevent, protect against, manage, or treat mast cell related disorders.

In some embodiments, an effective amount of an antibody provided herein is from about 0.01 mg to about 1,000 mg. Accordingly, the pharmaceutical composition of the present disclosure may include from about 0.001 mg, about 0.01 mg, about 0.05 mg, about 0.1 mg, about 0.5 mg, about 1 mg, about 5 mg, about 10 mg, about 15 mg, about 30 mg, about 50 mg, about 100 mg, about 200 mg, about 300 mg, about 400 mg, about 500 mg, about 600 mg, about 700 mg, about 800 mg, about 900 mg or about 1,000 mg of the anti-c-Kit antibody provided herein in a single dose, inclusive, including any values in between these numbers. In specific embodiments, an “effective amount” or “therapeutically effective amount” of an anti-c-Kit antibody described herein refers to an amount of an anti-c-Kit antibody described herein which is sufficient to achieve at least one, two, three, four or more of the following effects: the reduction or amelioration of the severity of mast cell related disorders and/or one or more symptoms associated therewith; the reduction in the duration of one or more symptoms associated with mast cell related disorders; the prevention in the recurrence of one or more symptoms of mast cell related disorders; the regression of mast cell related disorders and/or one or more symptoms associated therewith; the reduction in hospitalization of a subject; the reduction in hospitalization length; the increase in the survival of a subject with mast cell related disorders; the inhibition (e.g., partial inhibition) of the progression of mast cell related disorders and/or one or more symptoms associated therewith; the prevention of the development or onset of one or more symptoms associated mast cell related disorders; a decrease in the concentration of one or more inflammatory mediators (e.g., cytokines or interleukins) in biological specimens (e.g., plasma, serum, cerebral spinal fluid, urine, or any other biofluids) of a subject with mast cell related disorders; and improvement in the quality of life as assessed by methods well known in the art, e.g., questionnaires. In some embodiments, “effective amount” as used herein also refers to the amount of an antibody described herein to achieve a specified result (e.g., inhibition or suppression of one or more c-Kit biological activities of a cell, such as inhibition or suppression of proliferation, accumulation, migration, and/or degranulation of mast cell).

In some embodiments, an anti-c-Kit antibody described herein is administered as necessary, e.g., weekly, biweekly (i.e., once every two weeks), monthly, bimonthly, trimonthly, etc.

In some embodiments, a single dose of an anti-c-Kit antibody described herein is administered one or more times to a patient to impede, prevent, manage, treat, and/or ameliorate mast cell related disorders.

In particular embodiments, an anti-c-Kit antibody or pharmaceutical composition thereof is administered to a subject in accordance with the methods for treating mast cell related disorders, provided herein in cycles, wherein the anti-c-Kit antibody or pharmaceutical composition is administered for a period of time, followed by a period of rest (i.e., the anti-c-Kit antibody or pharmaceutical composition is not administered for a period of time).

The methods provided herein involve administering an anti-c-Kit antibody by any suitable routes. Non-limiting examples of routes of administration include, parenteral administration for example subcutaneous, intramuscular or intravenous administration, epidural administration, enteric administration, intracerebral administration, nasal administration, intraarterial administration, intracardiac administration, intraosseous infusion, intrathecal administration, and intraperitoneal administration. Methods provided herein include routes of administration targeting the brain, spinal cord, or ear or auricular tissue. In a particular embodiment, methods provided herein include routes of administration targeting the nervous system, e.g., central nervous system. The administration described herein can be subjected to the pharmaceutical composition of the present disclosure.

In specific embodiments, methods provided herein involve administering an anti-c-Kit antibody via a route suitable for crossing the blood-brain barrier.

### IV. Articles of Manufacture and Kits

In one embodiment of the present disclosure, also provided are kits or articles of manufacture comprising the pharmaceutical compositions described herein (e.g., anti-c-Kit antibody) in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

In some embodiments, kits may comprise compositions described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein. For example, in some embodiments, the kit comprises an anti-c-Kit antibody described herein, a pharmaceutically acceptable carrier suitable for administration, and one or more of: a buffer, a diluent, a filter, a needle, a syringe, and a package insert with instructions for performing administration.

### V. Antibody Production

Antibodies (e.g., human or humanized antibodies) described herein (or an antigen-binding fragment thereof) that immunospecifically bind to a c-Kit antigen (e.g., c-Kit epitope) can be produced by any method known in the art for the synthesis of antibodies, for example, by chemical synthesis or by recombinant expression techniques. The methods described herein employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

For example, humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28 (4/5): 489-498; Studnicka et al., 1994, Protein Engineering 7 (6): 805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13 (5): 353-60 (2000), Morea et al., Methods 20 (3): 267 79 (2000), Baca et al., J. Biol. Chem. 272 (16): 10678-84 (1997), Roguska et al., Protein Eng. 9 (10): 895 904 (1996), Couto et al., Cancer Res. 55 (23 Supp): 5973s-5977s (1995), Couto et al., Cancer Res. 55 (8): 1717-22 (1995), Sandhu J S, Gene 150 (2): 409-10 (1994), and Pedersen et al., J. Mol. Biol. 235 (3): 959-73 (1994). See also U.S. Patent Pub. No. US 2005/0042664 A1 (Feb. 24, 2005), which is incorporated herein by reference.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hy-bridomas 563-681 (Elsevier, N.Y., 1981). The term “monoclonal antibody” as used herein is not limited to antibodies produced through hybridoma technology. For example, monoclonal antibodies can be produced by recombinant technology, e.g., recombinant monoclonal antibodies expressed by a host cell, such as a mammalian host cell.

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the protein (e.g., extracellular domain of human c-Kit) used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). Additionally, a RIMMS (repetitive immunization multiple sites) technique can be used to immunize an animal (Kilptrack et al., 1997 Hybridoma 16:381-9, which is incorporated herein by reference).

Non-limiting examples of myeloma cell lines include murine myeloma lines, such as those derived from MOPC-21 and MPC-11 mouse tumors available from the Salk Institute Cell Distribution Center, San Diego, Calif., USA, and SP-2, or P3X63-Ag8.653 (ATCC CRL-1580) cells available from the American Type Culture Collection, Rockville, Md., USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Antibodies described herein include antibody fragments which recognize specific c-Kit antigens and can be generated by any technique known to those of skill in the art. For example, Fab and F(ab′)2 fragments described herein can be produced by pro-teolytic cleavage of immunoglobulin molecules, using enzymes such as papain (to produce Fab fragments) or pepsin (to produce F(ab′)2 fragments). A Fab fragment cor-responds to the two identical arms of an antibody molecule and contains the complete light chain paired with the VH and CHI domains of the heavy chain. A F(ab′)2 fragment contains the two antigen-binding arms of an antibody molecule linked by disulfide bonds in the hinge region.

In one embodiment, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1 (3): 253-263; Muylderman, 2001, J. Biotechnol. 74 (4): 277302; U.S. Pat. No. 6,005,079; and International Publication Nos. WO 94/04678, WO 94/25591, and WO 01/44301.

### EXAMPLE

The examples in this section are offered by way of illustration, and not by way of limitation.

### [Example 1] Preparation of Cell Line and Anti-c-Kit Antibody

### Cell Line and Culture

LAD2 is SCF-dependent human mast cell line with wild type c-Kit. Therefore, LAD2 cells are activated and proliferated in an SCF-dependent manner, like primary mast cells. Additionally, LAD2 expresses FcεRI, FcγRI, histamine, and tryptase, and is degranulated by the cross-linking of FcεRI or FcγRI, leading to the release of inflammatory mediators. Therefore, LAD2 is a suitable cell line for mast cell experiments (See Kirshenbaum A S, et al., (2003) Leuk Res 27:677-82).

LAD2 cells were cultured in StemPro-34 SFM (Thermo Fisher Scientific, MA, USA) with StemPro-34 nutrient supplement (2.5%, Thermo Fisher Scientific, MA, USA), L-glutamine (2 mM, Gibco, CA, USA), penicillin/streptomycin (1%, Hyclone, UT, USA), and recombinant human SCF (100 ng/mL, R&D Systems, MN, USA). Half of the medium was replaced weekly by adding an equal volume of fresh medium containing SCF. The cell density was maintained at 2 to 5×10⁵ cells/mL. The cells were incubated at 37° C. in 5% CO₂ incubator.

### Anti-C-Kit Antibodies

2G4 antibody was used to demonstrate that the antibodies binding to Ig-like domains D2 and/or D3 of c-Kit inhibit or suppress phosphorylation, proliferation, migration, etc. of the mast cells.

2G4 antibody used in the experiment was prepared in PATHEON BIOLOGICS (NJ) LLC (USA). 2G4 antibody binds to Ig-like domains D2 and/or D3 region of human c-Kit, specifically binds to at least one of R122, Y125, R181, K203, R205, S261, and H263 in the D2/3 region of c-Kit (Kim J O, et al., (2020) Int J Biol Macromol 159:66-78). Further, 2G4 antibody binds to c-Kit with high binding affinity (KD=2.83×10⁻¹² M), which is more than 500-fold stronger than that between SCF and c-Kit (KD=1.5×10⁻⁹ to 3×10⁻¹⁰ M; Debra D. Dahlen et al., Leukemia Research (2001); Tal Tilayov et al., Molecule, 2020).

4C9 antibody was prepared on a laboratory scale via the following method described in WO 2021/107566, and a sample with a final concentration of 5.31 mg/ml was used for the experiment.

A fully human 4C9 antibody targeting human c-Kit was produced as described previously (Kim J O, et al., (2020) Int J Biol Macromol 159:66-78). Briefly, a human recombinant c-Kit (Q26-T520, Elabscience, Wuhan, China) was immunized into humanized NSG mice (Orient Bio, Sungnam, Korea) implanted with human CD34+ hematopoietic stem cells (Lonza, Basel, Switzerland). The emulsion was produced by mixing the c-kit protein (1 μg/μL) with an equal volume of complete Freund's adjuvant (Sigma-Aldrich, St. Louis, MO, USA). Booster injections were administered during week 5. The human antibody titer in mouse serum was assessed using an indirect ELISA. Maxisorp plates were coated with the c-kit protein at 0.1 μg/well. Mice with a positive immune response were subjected to a final boost injection in week 7. Hy-bridomas with positive reactivity against c-kit in the ELISA were subcloned using a standard limiting dilution method. The antibody gene sequences of the heavy and light chain variable domains were determined by GenScript (Piscataway, NJ, USA). The nucleotide sequence of the 4C9 clone was codon optimized for Cricetulus griseus, synthesized as IgG1, and subcloned into the pCHO1.0 vector (Thermo Fisher Scientific, Waltham, MA, USA). The recombinant plasmids were transiently transfected into CHO-S cells cultured in CD CHO serum-free medium using the ExpiCHO™ Expression System Kit (Thermo Fisher Scientific, Waltham, MA, USA) for expression of the 4C9 antibody. The antibody was purified using Protein A Sepharose and SP Sepharose columns (Invitrogen, Carlsbad, CA, USA) as described in the Kim et al. (2020) article above.

The CDR sequences of the 2G4 antibody are as shown in Table 1, and the sequences of the heavy chain variable region, light chain variable region, heavy chain, and light chain are as shown in Table 2 and Table 3. The CDR sequences of the 4C9 antibody are as shown in Table 4, and the sequences of the heavy chain variable region and light chain variable region are as shown in Table 5.

TABLE 1

| CDR | Sequence | SEQ ID NO |
|---|---|---|
| H-CDR1 | GFTFSRYG | SEQ ID NO: 1 |
| H-CDR2 | IWYDGTNK | SEQ ID NO: 2 |
| H-CDR3 | AREDWAEAFD M | SEQ ID NO: 3 |
| L-CDR1 | QSLLHSNGYN Y | SEQ ID NO: 4 |
| L-CDR2 | LGS | SEQ ID NO: 5 |
| L-CDR3 | MQALQTIT | SEQ ID NO: 6 |

TABLE 2

| | Sequence | SEQ ID NO |
|---|---|---|
| Heavy chain | QVQLVESGGG VVQPGRSLRL | SEQ ID NO: 7 |
| variable | SCAASGFTFS RYGMHWVRQA | |
| region | PGKGLEWVAV IWYDGTNKDY | |
| | TDSVRGRFTI SRDNSKNTLY | |
| | LQMNSLRAED TAVYYCARED | |
| | WAEAFDMWGQ GTTVTVSS | |
| Light chain | DIVMTQSPLS LPVTPGEPAS | SEQ ID NO: 8 |
| variable | ISCRSSQSLL HSNGYNYLDW | |
| region | YLQKPGQSPQ LLIYLGSNRA | |
| | SGVPDRFSGS GSGTDFTLKI | |
| | SRVEAEDVGV YYCMQALQTI | |
| | TFGQGTRLEI K | |

TABLE 3

| | Sequence | SEQ ID NO |
|---|---|---|
| Heavy chain | QVQLVESGGG VVQPGRSLRL | SEQ ID NO: 9 |
| | SCAASGFTFS RYGMHWVRQA | |
| | PGKGLEWVAV IWYDGTNKDY | |
| | TDSVRGRFTI SRDNSKNTLY | |
| | LQMNSLRAED TAVYYCARED | |
| | WAEAFDMWGQ GTTVTVSSAS | |
| | TKGPSVFPLA PSSKSTSGGT | |
| | AALGCLVKDY FPEPVTVSWN | |
| | SGALTSGVHT FPAVLQSSGL | |
| | YSLSSVVTVP SSSLGTQTYI | |
| | CNVNHKPSNT KVDKKVEPKS | |
| | CDKTHTCPPC PA- PELLGGPS | |
| | FLFPPKPKD TLMISRTPEV | |
| | TCVVVDVSHE DPEVKFNWYV | |
| | DGVEVHNAKT KPREEQYNST | |
| | YRVVSVLTVL HQDWLNGKEY | |
| | KCKVSNKALP APIEKTISKA | |
| | KGQPREPQVY TLPPSRDELT KN- | |
| | QVSLTCLV KGFYPSDIAV | |
| | EWESNGQPEN NYKTTPPVLD | |
| | SDGSFFLYSK LTVDKSRWQQ | |
| | GNVFSCSVMH EALHNHYTQK | |
| | SLSLSPGK | |
| Light chain | DIVMTQSPLS LPVTPGEPAS | SEQ ID NO: 10 |
| | ISCRSSQSLL HSNGYNYLDW | |
| | YLQKPGQSPQ LLIYLGSNRA | |
| | SGVPDRFSGS GSGTDFTLKI | |
| | SRVEAEDVGV YYCMQALQTI | |
| | TFGQGTRLEI KRTVAAPSVF | |
| | IFPPSDEQLK SGTASVVCLL | |
| | NNFYPREAKV QWKVDNALQS | |
| | GNSQESVTEQ DSKDSTYSLS | |
| | STLTLSKADY EKHKVYACEV | |
| | THQGLSSPVT KSFNRGEC | |

TABLE 4

| CDR | Sequence | SEQ ID NO |
|---|---|---|
| H-CDR1 | SYYWS | SEQ ID NO: 15 |
| H-CDR2 | YIFYSGSTNY NPSLKS | SEQ ID NO: 16 |
| H-CDR3 | GYSSGWLDFH H | SEQ ID NO: 17 |
| L-CDR1 | RASQSISSYL N | SEQ ID NO: 18 |
| L-CDR2 | AASSLQS | SEQ ID NO: 19 |
| L-CDR3 | QQSYSTPIT | SEQ ID NO: 20 |

TABLE 5

| Variable | | |
|---|---|---|
| region | Sequence | SEQ ID NO |
| Heavy chain | QVQLQESGPG LVKPSETLSL | SEQ ID NO: 21 |
| | TCTVSGGSIG SYYWSWIRQP | |
| | PGKGLEWIGY IFYSGSTNYN | |
| | PSLKSRVTIS VDTSKNQFSL | |
| | KLSSVTAADT AVYYCARGYS SG- | |
| | WLDFHHWG QGTLVAVSS | |
| Light chain | DIQMTQSPSS LSASVGDRVT | SEQ ID NO: 22 |
| | ITCRASQSIS SYLNWYQQKP | |
| | GKAPKLLIYA ASSLQSGVPS | |
| | RFSGSGSGTD FTLTISSLQP | |
| | EDFATYYCQQ SYSTPITFGQ | |
| | GTRLEIK | |

### Statistical Analysis

In Examples 2-7 below, graphing and statistical analyses were performed using GraphPad Prism 5 (GraphPad Software, CA, USA). P-values were measured by two-tailed Student's t-test or one-way ANOVA with Dunnett's post-test. Statistical sig-nificance was set at P<0.05.

### [Example 2] c-Kit Antibodies Bind to LAD2 Cells

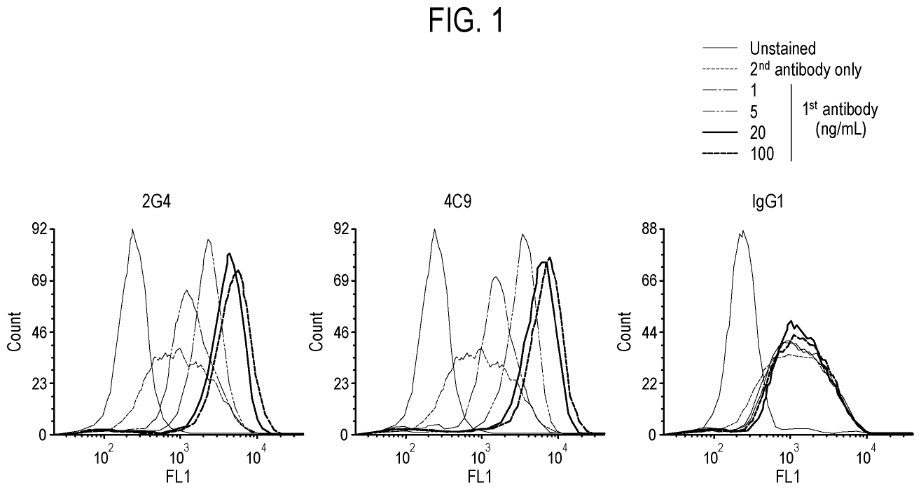

To demonstrate antibody binding to c-Kit on the cell surface of LAD2 cells, flow cytometry assay was performed. LAD2 cells were starved of SCF for 24 h, because SCF can cause internalization and degradation of c-Kit. Cells were rinsed with Dulbecco's phosphate-buffered saline (DPBS; Lonza, USA; Cat no.: 17-512Q) and blocked with PBS containing 5% bovine serum albumin (BSA) at 4° C. for 1 h. After blocking with BSA, Human BD Fc Block™ (2.5 μg/10⁶ cells, BD Biosciences, CA, USA) was treated to block binding of the antibody to the Fc receptor. The cells (2×10⁵ cells) were stained with 2G4, 4C9, or normal human IgG1 (Sino Biological, Beijing, China) at the indicated concentrations at 4° C. for 1 h. The cells were then rinsed thrice in PBS containing 2% BSA and stained with goat anti-human IgG secondary antibody (0.3 μg/mL, Invitrogen, CA, USA) at 4° C. for 1 h. After washing, the fluorescence signal was detected using CyFlow Cube6 (Sysmex Partec, Goerlitz, Germany), and the data analysis was performed using FCS Express 6 Flow (De Novo software, CA, USA). The results of flow cytometry analysis are shown in FIG. 1.

According to FIG. 1, both 2G4 and 4C9 antibodies bound to LAD2 cells in a dose-dependent manner, and the fluorescence signal was saturated at a concentration of 100 ng/mL. However, the binding signal of normal human IgG1 did not increase, except for the non-specific signal by the secondary antibody. These results demonstrate that both 2G4 antibody and 4C9 antibody immunospecifically bind to c-Kit present on LAD2 cells, specifically the extracellular domain of c-Kit in that 2G4 binds to D2 and/or D3 region of human c-Kit and 4C9 to D1 and/or D2 region of human c-Kit.

### [Example 3] an Exemplary Anti-c-Kit Antibody of the Present Disclosure Inhibits SCF/c-Kit Signaling

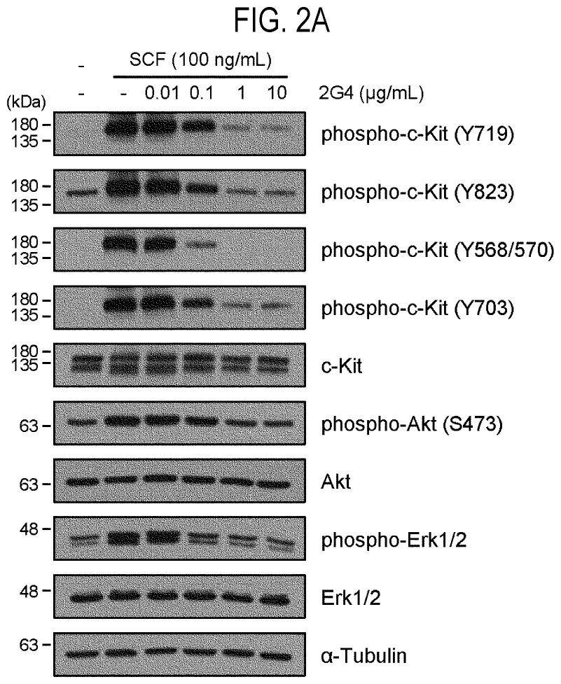

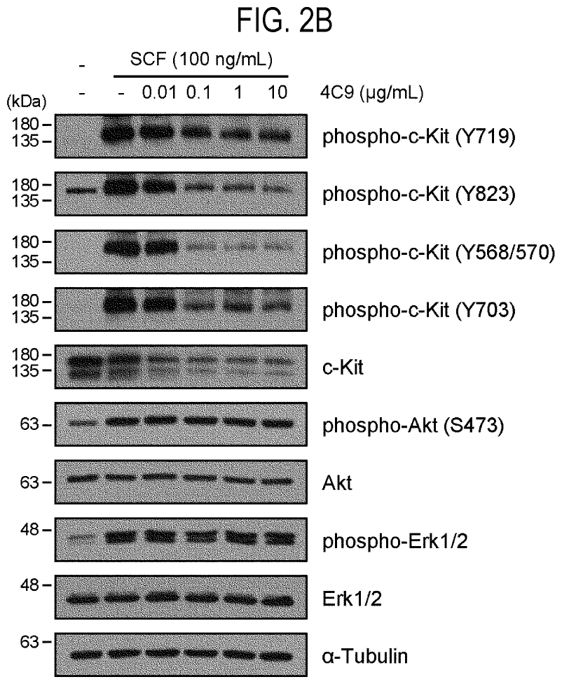

To verify whether 2G4 and 4C9 antibodies can inhibit SCF-mediated c-Kit activation, LAD2 cells were seeded into a 6-well culture plate (1×10⁶ cells/well) in SCF-deficient medium for 24 h. After SCF-starvation, LAD2 cells were pretreated with 2G4 or 4C9 antibody at the indicated concentrations at 37° C. for 1 h. The cells were then stimulated with 100 ng/mL SCF for an additional 10 min. Thereafter, the cells were lysed in RIPA lysis buffer (pH 7.6, 20 mM Tris-HCl, 150 mM NaCl, 1 mM Na₂EDTA, 1 mM EGTA, 1% NP-40, 1% sodium deoxycholate, 0.1% SDS, 10 mM β-glycerophosphate, 1 mM Na₃OV₄, 10 mM NaF, 1 μg/mL leupeptin, 1 mM PMSF, 5 μg/mL aprotinin, and 2 mM 2-mercaptoethanol). Phosphorylation of c-Kit and its downstream signaling molecules (Akt and Erk) was analyzed by western blotting. α-Tubulin was used as a loading control. The antibodies used herein were anti-phospho-c-Kit (Tyr719, Tyr823, Tyr568/570, and Tyr703, Cell Signaling Technology, MA, USA), anti-phospho Akt (Ser473, Cell Signaling Technology, MA, USA), anti-phospho-Erk1/2 (Cell Signaling Technology, MA, USA), anti-c-Kit (R&D Systems, MN, USA), anti-Akt (Santa Cruz Biotechnology, CA, USA), anti-Erk1/2 (Santa Cruz Biotechnology, CA, USA), and anti-α-tubulin (laboratory-made). The results of western blot analysis are shown in FIGS. 2a and 2b.

Pre-treatment with 2G4 antibody inhibited c-Kit phosphorylation induced by SCF in a dose-dependent manner (FIG. 2a). 2G4 antibody inhibited phosphorylation at all tested tyrosine residues of c-Kit (Y719, Y823, Y568/570, and Y703). In particular, at concentrations of 1 μg/ml or higher, 2G4 antibody inhibited most of phosphorylation. In addition, phosphorylation of Akt and Erk1/2 which are downstream signals of c-Kit decreased by 2G4 antibody in a dose-dependent manner. These results demonstrates that 2G4 antibody effectively inhibited the activation of c-Kit signaling induced by SCF in LAD2 cells.

However, while 4C9 antibody significantly downregulated the total c-Kit expression in a dose-dependent manner, it did not inhibit the phosphorylation of Akt and Erk1/2 (FIG. 2b), which are related to the downstream signaling pathways of c-Kit phosphorylation. These results suggest that 4C9 antibody only inhibits the expression of c-Kit itself, and does not inhibit c-Kit phosphorylation by the binding of c-Kit and SCF.

This also suggests that not all anti-c-Kit antibodies inhibit phosphorylation of mast cells and demonstrates that the anti-c-Kit antibody of the present disclosure, which binds to Ig-like domain D2 and/or D3 of c-Kit with high binding affinity, effectively inhibit phosphorylation of mast cells.

### [Example 4] an Exemplary Anti-c-Kit Antibody of the Present Disclosure Suppresses Cell Proliferation of Mast Cells

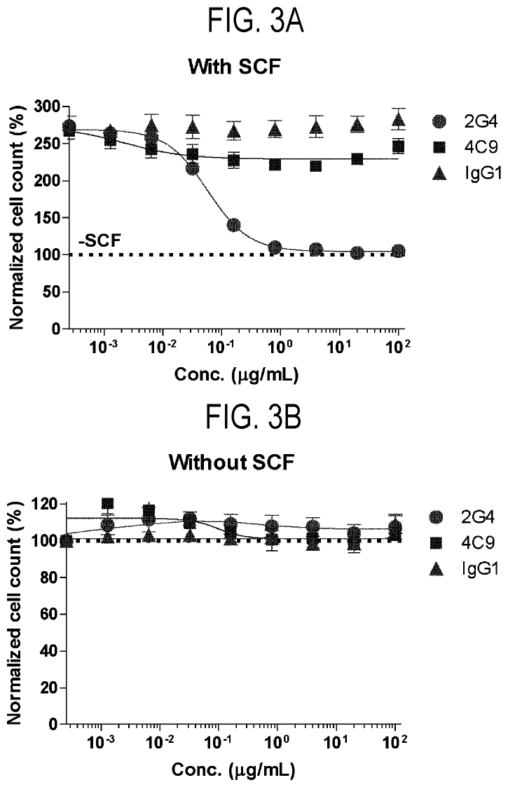

LAD2 cells were seeded into 96-well culture plates (1×10⁴ cells/well), with or without 100 ng/mL SCF. The cells were incubated with serial 5-fold of 2G4, 4C9, or normal human IgG1 at a final concentration 100 μg/mL at 37° C. for 7 days. The cells were then stained with 10 μM Hoechst 33342 (Thermo Fisher scientific, MA, USA) for 30 min, and counted using Celigo imaging cytometer (Nexcelom, MA, USA). The results of cell proliferation assay are shown in FIGS. 3a and 3b.

In the cell proliferation assay, the number of LAD2 cells was increased by 100 ng/mL SCF by more than 2.5-fold for 7 days (comparing the normalized cell counts in the well without SCF (black dashed line) and in the well with SCF and without the antibodies in FIG. 3a). SCF-mediated proliferation was potently suppressed by 2G4 antibody in a dose-dependent manner, but not by 4C9 or normal human IgG1 (FIG. 3a). The half-maximal inhibitory concentration (IC₅₀) value of 2G4 antibody against LAD2 cells was 0.058 μg/mL, and the proliferation was completely inhibited by 2G4 antibody at concentrations above 0.8 μg/mL. In the absence of SCF, LAD2 cells did not proliferate and 2G4, 4C9, and normal human IgG1 had no effect on cell proliferation at all (FIG. 3b). This implied that 2G4 antibody does not directly induce cell death against LAD2 but can inhibit proliferation mediated by SCF.

Taken together with the results of Example 3, these results demonstrate that the anti-c-Kit antibody of the present disclosure, e.g. 2G4 antibody, which binds to Ig-like domains D2 and/or D3 of c-Kit and inhibits the c-Kit phosphorylation by SCF binding, can suppress the SCF-mediated proliferation of the mast cells. However, 4C9 antibody which does not inhibit the c-Kit phosphorylation by SCF binding (i.e. downstream of c-Kit signaling pathway) does not suppress SCF-mediated proliferation of the mast cells.

This also suggests that not all anti-c-Kit antibodies suppress proliferation of mast cells and demonstrates that the anti-c-Kit antibody of the present disclosure, which binds to Ig-like domain D2 and/or D3 of c-Kit with high binding affinity, effectively suppresses proliferation of mast cells.

This also suggests that not all anti-c-Kit antibodies suppress proliferation of mast cells and demonstrates that the anti-c-Kit antibody of the present disclosure, which binds to Ig-like domain D2 and/or D3 of c-Kit with high binding affinity, effectively suppresses proliferation of mast cells.

### [Example 5] an Exemplary Anti-c-Kit Antibody of the Present Disclosure Suppresses Migration of Mast Cells

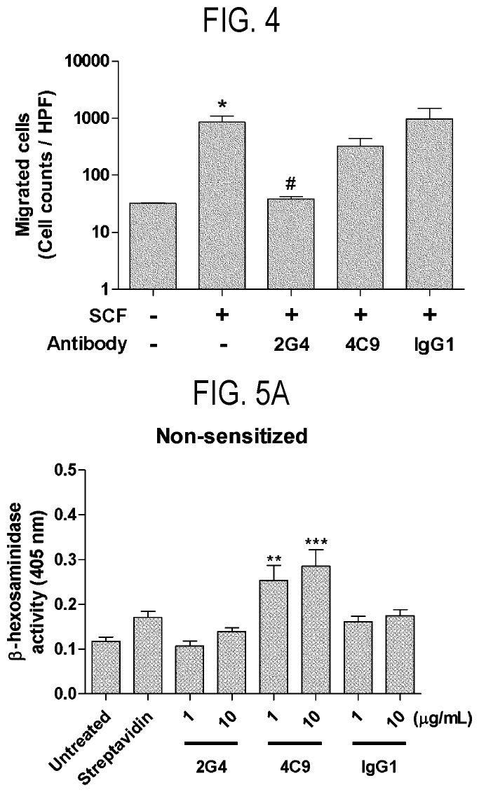

LAD2 cells were SCF-starved for 24 h. Thereafter, 1×10⁶ cells were seeded into the upper chamber of a 6-transwell plate with 8 μm pores (Costar, MA, USA) with low-supplement medium (StemPro-34 SFM with 0.5% StemPro-34 nutrient supplement, 2 mM L-glutamine, and 1% penicillin/streptomycin). Next, 1 μg/mL of 2G4, 4C9, or normal human IgG1 was added to the upper chamber, and 100 ng/mL SCF was added to the lower chamber for 24 h. After removing the upper chamber, migrated cells in the lower chamber were microscopically counted using high-power field (HPF, ×40 mag-nification) in five different fields. The results of cell migration assay are shown in FIG. 4.

In the migration assay using a transwell plate with 8 μm pore polycarbonate membrane, the number of cells passing through the membrane increased more than 26-fold by 100 ng/mL SCF (FIG. 4). The increase in migration induced by SCF was completely suppressed by 1 μg/mL of 2G4 antibody at the basal level. However, LAD2 migration was partially inhibited by 4C9 antibody, but no significant differences were observed (P=0.1071). There was no decrease in migration with the presence of normal human IgG1.

Similar to the results of Example 4, the anti-c-Kit antibody of the present disclosure, i.e. 2G4 antibody, which binds to Ig-like domains D2 and/or D3 of c-Kit and inhibits the c-Kit phosphorylation by SCF binding, can suppress the migration of the mast cells by SCF. However, 4C9 antibody which does not inhibit the c-Kit phosphorylation by SCF binding (i.e. downstream of c-Kit signaling pathway) does not suppress the migration of the mast cells by SCF.

This also suggests that not all anti-c-Kit antibodies suppress migration of mast cells and demonstrates that the anti-c-Kit antibody of the present disclosure, which binds to certain Ig-like domain of c-Kit with high binding affinity, effectively suppresses migration of mast cells.

The mechanism of action governing inhibition of mast cell proliferation and migration is a unique activity of the anti-c-Kit antibody of the present disclosure, which differs from conventional therapeutics, including antihistamines, corticosteroids, and anti-IgE antibodies. The anti-c-Kit antibody of the present disclosure may thus exhibit an effective therapeutic effect on mast cell related disorder (e.g., mastocytosis, MCAS and the like) which may not be sufficiently treated from conventional therapeutics. In contrast, 4C9 antibody, which does not have the characteristics of the anti-c-Kit antibody of the present disclosure, did not sufficiently inhibit the proliferation and migration of LAD2 cells.

### [Example 6] an Exemplary Anti-c-Kit Antibody of the Present Disclosure does not Induce Mast Cell Degranulation

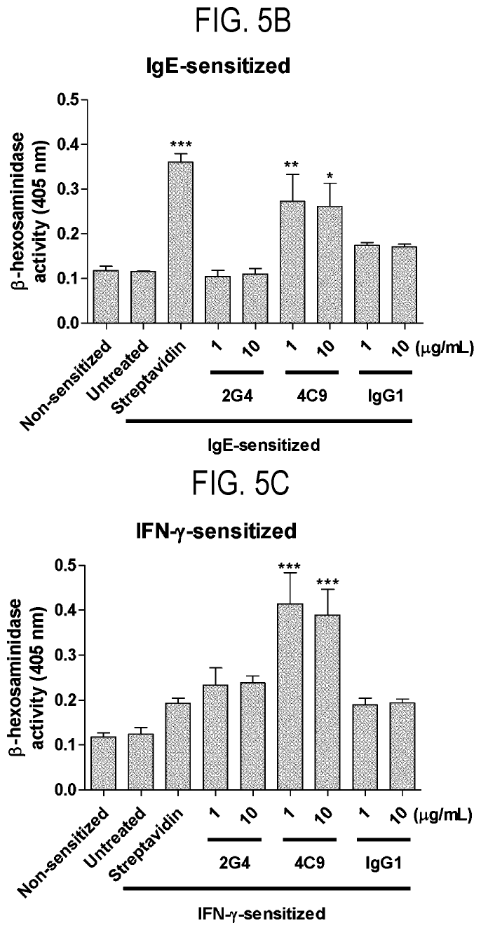

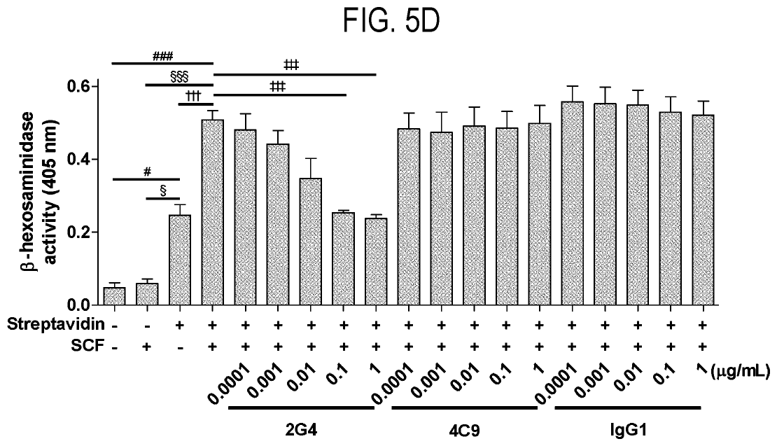

Human mast cells express FcγRs, including FcγRI (CD64) and FcγRII (CD32), but not FcγRIII (CD16). Binding of the Fc region of IgG to FcγRs may trigger degranulation which can cause hypersensitivity reactions (HSRs) in patients receiving therapeutic antibodies. Moreover, multimeric IgG-antigen immune complexes can bind to FcγRs with high-avidity interactions. Therefore, β-hexosaminidase assay was conducted to determine whether 2G4 or 4C9 antibody increase degranulation in LAD2 cells. Non-sensitized, IgE-sensitized, and interferon (IFN)-γ-sensitized LAD2 cells were prepared. Biotinylated human IgE (200 ng/mL, NBS-C Bioscience, Vienna, Austria) or IFN-γ (150 ng/mL, PeproTech, NJ, USA) was added to the cells at 37° C. for 24 h (in SCF-deficient medium). After sensitization, the cells were washed twice with HEPES buffer (10 mM HEPES, 137 mM NaCl, 2.7 mM KCl, 0.4 mM Na₂HPO₄, 5.6 mM glucose, 1.8 mM CaCl₂), 1.3 mM MgSO₄, and 0.04% BSA) and seeded onto a 96-well culture plate (10,000 cells/well). The cells were then incubated with 1 or 10 μg/mL of 2G4, 4C9, or normal human IgG1 at 37° C. (without CO₂) for 1 h. Streptavidin (2 ng/mL, Sigma-Aldrich, MO, USA), as a positive control, was used to crosslink biotinylated-IgE. After centrifugation at 450×g for 5 min, 50 μL of supernatant was added to 100 μL p-nitrophenyl N-acetyl-β-D-glucosamide (PNAG) solution (3.5 mg/mL, Sigma-Aldrich, MO, USA) at 37° C. (without CO₂) for 1.5 h. When 50 μL of glycine buffer (0.4 M) was added, the yellow color indicated β-hexosaminidase activity (See, e.g., Kuehn H S, Radinger M and Gilfillan A M (2010) Measuring mast cell mediator release. Curr Protoc Immunol Chapter 7: Unit7 38). Ab-sorbance at 405 nm wavelength was measured using SPECTROstar Nano microplate reader (BMG Labtech, Oetenberg, Germany). These results are shown in FIGS. 5a to 5c.

Further, to examine whether 2G4, 4C9, or normal human IgG1 could suppress degranulation synergistically increased by SCF, IgE-sensitized LAD2 cells were re-suspended in HEPES buffer and seeded onto a 96-well culture plate (10,000 cells/well). Cells were pretreated with 2G4, 4C9, or normal human IgG1 at 37° C. for 0.5 h, and treated with 100 ng/mL SCF for an additional 0.5 h. Thereafter, streptavidin was then added to crosslink biotinylated-IgE for 0.5 h and β-hexosaminidase release assay was carried out. These results are shown in FIG. 5d.

1. An Exemplary Anti-c-Kit Antibody of the Present Disclosure does not Induce Antibody Mediated Mast Cell Degranulation

There was no significant increase in degranulation mediated by 2G4 antibody and normal human IgG1 in non-sensitized LAD2 cells, but 4C9 antibody increased degranulation in a dose-dependent manner, compared with untreated cells (FIG. 5a).

In IgE-sensitized cells, 2G4 antibody did not increase degranulation, whereas 4C9 antibody significantly increased degranulation (FIG. 5b). Normal human IgG1 slightly increased degranulation; however, the difference was not statistically significant (P>0.5808). Streptavidin for IgE ligation as mimetics of allergen, was used as a positive control to demonstrate activity of mast cell degranulation. Streptavidin increased the degranulation more than 3-fold compared to untreated cells.

It has been reported that IFN-γ strongly increases the expression of FcγRI in human mast cells, and degranulation may be enhanced by FcγRI [Woolhiser M R et al., Clin Immunol 110:172-80]. Thus, addition of IFN-γ in the mast cell degranulation assay may mimic the response in activated immune system in human. In IFN-γ-sensitized cells, 2G4 antibody (P>0.1784) and normal human IgG1 (P>0.6074) slightly increased degranulation, but the difference was not statistically significant (FIG. 5c). However, 4C9 antibody significantly increased the degranulation, compared to degranulation in non- or IgE-sensitized cells.

It thus shows that the anti-c-Kit antibody of the present disclosure does not induce antibody mediated mast cell degranulation and thus can be safely used when administered to a patient, for example, by not causing HSRs.

2. An Exemplary Anti-c-Kit Antibody of the Present Disclosure Inhibits Mast Cell Degranulation, which is Synergistically Increased by SCF

SCF is known to increase IgE-mediated degranulation in LAD2 cells. In the mast cell degranulation assay (FIG. 5d), SCF alone did not significantly increase secretion of β-hexosaminidase in IgE sensitized LAD2 cells, whereas streptavidin (allergen mimetics), a biotinylated-IgE crosslinker, increased secretion of β-hexosaminidase by 3-fold. A treatment of SCF and streptavidin together showed synergistic effect as the treatment of SCF and streptavidin increased secretion of β-hexosaminidase by 2-fold compared to streptavidin alone.

The increase in degranulation by SCF and streptavidin was inhibited by 2G4 antibody in a dose-dependent manner (IC₅₀=0.00636 μg/mL), and the degranulation decreased (to the degranulation level in the streptavidin+/SCF sample) at the 2G4 antibody concentrations above 0.1 μg/mL. However, 4C9 antibody and normal human IgG1 did not inhibit degranulation at all. This result indicated that 2G4 antibody can inhibit the excessive increase in mast cell degranulation by SCF.

Most mast cell related diseases are caused by IgE-mediated degranulation. According to the results above, 2G4 antibody inhibited an increase in degranulation of mast cells by IgE-ligation and SCF. However, 4C9 antibody did not reduce the IgE-mediated degranulation of mast cells.

This also suggests that not all anti-c-Kit antibodies inhibit IgE-mediated degranulation of mast cells and demonstrates that the anti-c-Kit antibody of the present disclosure, which binds to certain Ig-like domain of c-Kit with high binding affinity, effectively inhibits IgE-mediated degranulation of mast cells.

### [Example 7] an Exemplary Anti-c-Kit Antibody of the Present Disclosure Inhibits SCF-Mediated Modulation of Cytokine Secretion by Mast Cells

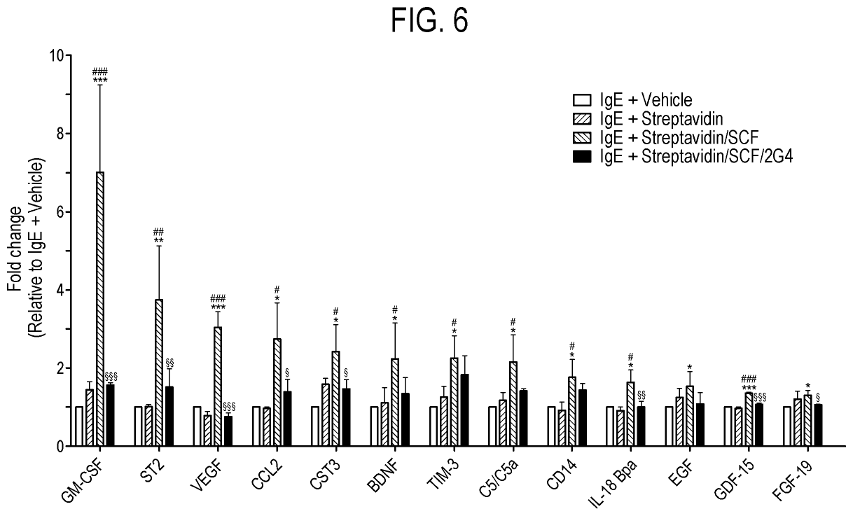

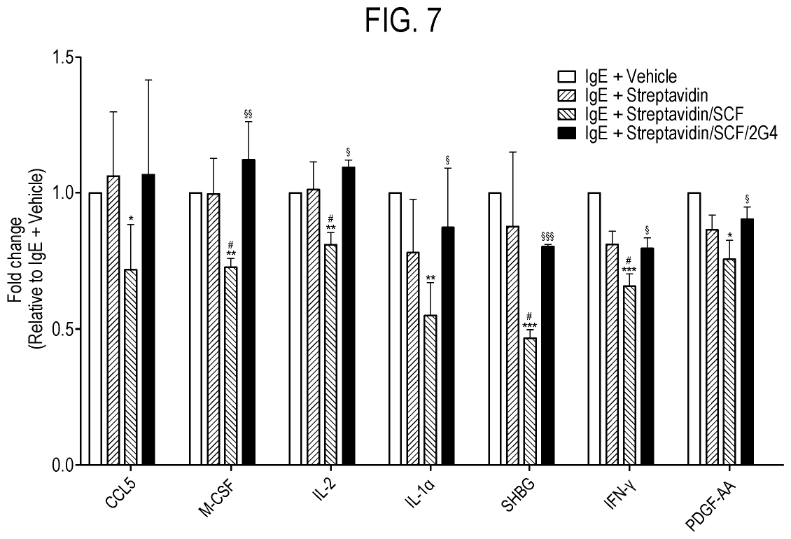

LAD2 cells (1×10⁶ cells/mL) were sensitized with biotinylated-IgE (200 ng/mL) at 37° C. for 24 h. The cells were then washed twice with PBS and seeded into a 12-well culture plates (1×10⁶ cells/well) with low-supplement medium. Thereafter, 2G4 antibody (1 μg/mL), SCF (100 ng/ml), and streptavidin (10 ng/mL) were added to the cells at intervals of 0.5 h. After 24 h of incubation, the cell-culture supernatant was harvested, and the cytokines released by LAD2 cells were blotted using the Human XL cytokine array kit (R&D Systems, MN, USA) according to the manufacturer's protocol. The intensity of each blot was measured using the ImageJ software (US National In-stitutes of Health, MD, USA). All experiments were independently repeated thrice. The results of cytokine-release assay are shown in FIGS. 6 and 7.

The level of various cytokines significantly increased following treatment with 100 ng/mL SCF (FIG. 6). The increase in the level of these cytokines was potently inhibited by treatment with 1 μg/mL of 2G4 antibody. In particular, GM-CSF is increased by SCF by more than 7-fold, but the increase is completely reduced by 2G4 antibody to the basal level (FIG. 6). In addition, suppression of tumorigenicity 2 (ST2), vascular endothelial growth factor (VEGF), C—C motif chemokine ligand 2 (CCL2, also known MCP-1), cystatin C (CST3), brain-derived neurotrophic factor (BDNF), T cell immunoglobulin and mucin-domain containing-3 (TIM-3), and complement component C5/C5a increased more than 2-fold, but all were effectively reduced by 2G4 antibody (FIG. 6).

Meanwhile, a few cytokines, including CCL5, macrophage colony-stimulating factor (M-CSF), and interleukin-2 (IL-2), were downregulated by SCF (FIG. 7). The downregulation was also suppressed by 2G4 antibody.

Collectively, SCF induces the modulation of cytokine secretion by mast cells, and the modulation is mainly shown by an increase in pro-inflammatory cytokines that exacerbate mast cell diseases. The anti-c-Kit antibody of the present disclosure inhibited the modulation of cytokines and can be used as a therapeutic agent for the treatment of mast cell diseases.

The above results showed that the anti-c-Kit antibody of the present disclosure, which binds to a human c-Kit epitope at a particular location, is effective for treatment, prevention, management, and/or amelioration of mast cell disorder and its symptoms. Particularly, the results of Example show that the anti-c-Kit antibody of the present disclosure such as 2G4 antibody has potential as a therapeutic agent for mast cell related disorders, with a mechanism different from that of conventional therapeutics. The anti-c-Kit antibody of the present disclosure binds to c-Kit with high affinity and completely blocks the binding of SCF, a ligand of c-Kit. Blockade of SCF/c-Kit signaling effectively inhibits cell proliferation, migration, degranulation, and cytokine release in human mast cells. Therefore, these results suggest that the anti-c-Kit antibody of the present disclosure exhibits therapeutic activity and effect for mast cell diseases.

From the above descriptions, a person skilled in the art to which the present disclosure pertains will understand that the present disclosure can be practiced in other specific forms without changing its technical idea or essential features. In this regard, it is understood that the examples described above are exemplary and not limiting in all respects. The scope of the present disclosure is construed so as to include the meaning and scope of the claims and all modifications, alterations or alternatives derived from the present disclosure and its equivalent concepts.

### (Sequence List)

Amino acid sequence of L-CDR2 of 2g4 antibody: LGS (SEQ ID NO: 5)

1. A method of treating, preventing, managing or ameliorating one or more mast cell related disorders in a subject,
comprising administering to a subject in need thereof a therapeutically effective amount of an anti-c-Kit antibody which specifically binds to human c-Kit epitope comprising an amino acid sequence of SEQ ID No: 13, or an antigen binding fragment thereof.

2. The method of claim 1, wherein the anti-c-Kit antibody or the antigen binding fragment thereof specifically binds to human c-Kit epitope comprising an amino acid sequence of SEQ ID Nos: 11 and 12.

3. The method of claim 1, wherein the anti-c-Kit antibody or the antigen binding fragment thereof specifically binds to at least one of R122, Y125, R181, K203, R205, S261 and H263 on amino acid sequence of SEQ ID NO: 14.

4. The method of claim 1, wherein the anti-c-Kit antibody or the antigen binding fragment thereof specifically binds to c-Kit with equilibrium dissociation constant (KD) value less than or equal to 10⁻⁸ M, 10⁻⁹ M, 10⁻¹⁰ M, 10⁻¹¹ M, 8×10⁻¹² M, 6×10⁻¹² M, 4×10⁻¹² M, 3×10⁻¹² M, 2×10⁻¹² M, 1×10⁻¹² M, or 10⁻¹³ M.

5. The method of claim 1, wherein the anti-c-Kit antibody or the antigen binding fragment thereof specifically binds to c-Kit with binding affinity which is more than 50-fold stronger, 100-fold stronger, 150-fold stronger, 200-fold stronger, 250-fold stronger, 300-fold stronger, 350-fold stronger, 400-fold stronger, 450-fold stronger, 500-fold stronger, 550-fold stronger, or 600-fold stronger than that between SCF and c-Kit.

6. A method of treating, preventing, managing or ameliorating one or more mast cell related disorders in a subject,
comprising administering to a subject in need thereof a therapeutically effective amount of an anti-c-Kit antibody or an antigen binding fragment thereof, wherein the antibody anti-c-Kit antibody or the antigen binding fragment thereof cross-competes for binding to human c-Kit epitope on an amino acid sequence of SEQ ID Nos: 11 and 12 with a reference antibody comprising a VH CDR1 of SEQ ID NO: 1, a VH CDR2 of SEQ ID NO: 2, a VH CDR3 of SEQ ID NO: 3, a VL CDR1 of SEQ ID NO: 4, a VL CDR2 of SEQ ID NO: 5 and a VL CDR3 of SEQ ID NO: 6.

7. A method of treating, preventing, managing or ameliorating one or more mast cell related disorders in a subject,
comprising administering to a subject in need thereof a therapeutically effective amount of an anti-c-Kit antibody or an antigen binding fragment thereof comprising a VH CDR1 of SEQ ID NO: 1, a VH CDR2 of SEQ ID NO: 2, a VH CDR3 of SEQ ID NO: 3, a VL CDR1 of SEQ ID NO: 4, a VL CDR2 of SEQ ID NO: 5 and a VL CDR3 of SEQ ID NO: 6, or comprises a variant of the CDRs,
wherein the variant of the CDRs comprises modification, deletion or substitution of one, two, three, four, or five amino acid(s) present in at least one CDR.

8. The method of claim 7, wherein the anti-c-Kit antibody or the antigen binding fragment thereof comprises a VH CDR1 of SEQ ID NO: 1, a VH CDR2 of SEQ ID NO: 2, a VH CDR3 of SEQ ID NO: 3, a VL CDR1 of SEQ ID NO: 4, a VL CDR2 of SEQ ID NO: 5 and a VL CDR3 of SEQ ID NO: 6.

9. The method of claim 8, wherein the anti-c-Kit antibody or the antigen binding fragment thereof comprises a VH comprising an amino acid sequence having at least 95%, 96%, 97%, 98% or 99% sequence identity to SEQ ID NO: 7 and a VL comprising an amino acid sequence having at least 95%, 96%, 97%, 98% or 99% sequence identity to SEQ ID NO: 8.

10. The method of claim 9, wherein the anti-c-Kit antibody or the antigen binding fragment thereof comprises a VH comprising SEQ ID NO: 7 and a VL comprising SEQ ID NO: 8.

11. The method of claim 10, wherein the anti-c-Kit antibody or the antigen binding fragment thereof comprises a heavy chain comprising SEQ ID NO: 9 and a light chain comprising SEQ ID NO: 10.

12. The method of claim 1, wherein the anti-c-Kit antibody is a bivalent monospecific antibody.

13. The method of claim 1, wherein the anti-c-Kit antibody is a humanized antibody.

14. The method of claim 1, wherein the anti-c-Kit antibody is a naked antibody.

15. The method of claim 1, wherein the subject is a human.

16. The method of claim 1, wherein the therapeutically effective amount of the anti-c-Kit antibody or the antigen binding fragment thereof is about 0.01 mg/kg to 1,000 mg/kg.

17. The method of claim 1, wherein the mast cell related disorders are selected from the group consisting of mast cell activation syndrome (MCAS), primary mast cell activation syndrome, monoclonal mast cell activation syndrome (MMAS), idiopathic mast cell activation syndrome, secondary mast cell activation syndrome, asthma, allergic rhinitis, allergic inflammation, food allergies, rheumatoid arthritis, inflammatory bowel disease (IBD), multiple sclerosis, anaphylaxis, idiopathic anaphylaxis, Ig-E mediated anaphylaxis, non-Ig-E mediated anaphylaxis, neurofibromatosis, atopic dermatitis, psoriasis, urticaria, idiopathic urticaria, chronic urticaria, chronic spontaneous urticaria, allergic urticaria, idiopathic urticaria, cold induced urticaria, heat induced urticaria, dermatographic urticaria, vibratory urticaria, cholinergic urticaria, contact urticaria, symptomatic dermatographism, normo complementemic urticarial vasculitis, hypereosinophilic syndrome, fibrosis, idiopathic pulmonary fibrosis (IPF), pulmonary fibrosis, hepatic fibrosis, cardiac fibrosis, scleroderma, myelofibrosis, inflammatory conditions, pulmonary arterial hypertension (PAH), irritable bowel syndrome (IBS), dermatosis, mast cell activation disorder (MCAD), mastocytosis, cutaneous mastocytosis (e.g., urticaria pigmentosa), indolent systemic mastocytosis (ISM), smoldering systemic mastocytosis (SSM), mastocytosis with an associated hematologic neoplasm (SM-AHN), aggressive systemic mastocytosis (ASM), mast cell leukemia (MCL), mast cell sarcoma, systemic mastocytosis (SM), advanced SM (AdvSM), non-advanced SM (non-Adv SM), swelling (angioedema), primary pulmonary hypertension (PPH), mastocytic gastroenteritis, mastocytic colitis, pruritus, chronic pruritis, pruritis secondary to chronic kidney failure and heart, heart, vascular, intestinal, brain, kidney, liver, pancreas, muscle, bone and skin conditions associated with mast cells, autoimmune diseases, respiratory diseases, allergic diseases (including, for example. food allergies), allergic sinusitis, allergic contact dermatitis, erythema nodosum, erythema multiforme, cutaneous necrotizing venulitis and insect bite skin inflammation, bronchial asthma, inflammatory diseases, diabetes, type I diabetes, type II diabetes, central nervous system (CNS) disorders, interstitial cystitis and hematologic disorders.

18. The method of claim 1, wherein the mast cell related disorders are selected from the group consisting of chronic spontaneous urticaria, cold induced urticaria, symptomatic dermatographism and normo complementemic urticarial vasculitis.

19. The method of claim 1, a route of administration of the anti-c-Kit antibody or the antigen binding fragment thereof is at least one of oral administration, subcutaneous administration, intramuscular administration intravenous administration, epidural administration, enteric administration, intracerebral administration, nasal administration, intraarterial administration, intracardiac administration, intraosseous infusion, intrathecal administration, and intraperitoneal administration.

20.-22. (canceled)


SEQUENCE LISTING (22 sequences)

SEQ ID NO: 1 - protein, 8 aa, synthetic construct
GFTFSRYG

SEQ ID NO: 2 - protein, 8 aa, synthetic construct
IWYDGTNK

SEQ ID NO: 3 - protein, 11 aa, synthetic construct
AREDWAEAFDM

SEQ ID NO: 4 - protein, 11 aa, synthetic construct
QSLLHSNGYNY

SEQ ID NO: 5 - skipped

SEQ ID NO: 6 - protein, 8 aa, synthetic construct
MQALQTIT

SEQ ID NO: 7 - protein, 118 aa, synthetic construct
QVQLVESGGGVVQPGRSLRLSCAASGFTFSRYGMHWVRQAPGKGLEWVAVIWYDGTNKDYTDSVRGRFTISRDNSKNTLYLQMNSLRAEDTAVYYCAREDWAEAFDMWGQGTTVTVSS

SEQ ID NO: 8 - protein, 111 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTITFGQGTRLEIK

SEQ ID NO: 9 - protein, 448 aa, synthetic construct
QVQLVESGGGVVQPGRSLRLSCAASGFTFSRYGMHWVRQAPGKGLEWVAVIWYDGTNKDYTDSVRGRFTISRDNSKNTLYLQMNSLRAEDTAVYYCAREDWAEAFDMWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 84 aa, synthetic construct
RSLYGKEDNDTLVRCPLTDPEVTNYSLKGCQGKPLPKDLRFIPDPKAGIMIKSVKRAYHRLCLHCSVDQEGKSVLSEKFILKVR

SEQ ID NO: 12 - protein, 24 aa, synthetic construct
SSVYSTWKRENSQTKLQEKYNSWH

SEQ ID NO: 13 - protein, 197 aa, synthetic construct
DPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVD

SEQ ID NO: 14 - protein, 284 aa, synthetic construct
  SITE 1: note = Q26 of human c-Kit domain D1-D3
  SITE 284: note = D309 of human c-Kit domain D1-D3
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVD

SEQ ID NO: 15 - protein, 5 aa, synthetic construct
SYYWS

SEQ ID NO: 16 - protein, 16 aa, synthetic construct
YIFYSGSTNYNPSLKS

SEQ ID NO: 17 - protein, 11 aa, synthetic construct
GYSSGWLDFHH

SEQ ID NO: 18 - protein, 11 aa, synthetic construct
RASQSISSYLN

SEQ ID NO: 19 - protein, 7 aa, synthetic construct
AASSLQS

SEQ ID NO: 20 - protein, 9 aa, synthetic construct
QQSYSTPIT

SEQ ID NO: 21 - protein, 119 aa, synthetic construct
QVQLQESGPGLVKPSETLSLTCTVSGGSIGSYYWSWIRQPPGKGLEWIGYIFYSGSTNYNPSLKSRVTISVDTSKNQFSLKLSSVTAADTAVYYCARGYSSGWLDFHHWGQGTLVAVSS

SEQ ID NO: 22 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIK